(19) United States
(12) Patent Application Publication
(10) Pub. No.: US 2022/0338892 A1
(43) Pub. Date: Oct. 27, 2022

(54) MINIATURIZED INTRA-BODY CONTROLLABLE MEDICAL DEVICE

(71) Applicant: Miraki Innovation Think Tank, LLC, Cambridge, MA (US)

(72) Inventors: Santosh Iyer, Somerville, MA (US); Adeel Saleem Shafi, Cambridge, MA (US); Christopher J. Velis, Lexington, MA (US)

(21) Appl. No.: 17/763,665

(22) PCT Filed: Sep. 25, 2020
(86) PCT No.: PCT/US2020/052925

§ 371 Date: Mar. 25, 2022

Related U.S. Application Data
(60) Provisional application No. 62/906,415, filed on Sep. 26, 2019.

(51) Int. Cl. A61B 17/29

(52) U.S. Cl. CPC A61B 17/29; A61B 2017/2927; A61B 2017/00398

(57) ABSTRACT

Systems and methods are disclosed for medical devices which can operate within a person in connection a medical procedure. In aspects of the present disclosure, a system for assisting with a surgical procedure includes a swarm of medical devices sized to be wholly deployed within a surgical site of a patient where the swarm of medical devices is configured to operate concurrently within the patient to assist a surgeon to perform a surgical procedure in the patient. The swarm of medical devices includes a first medical device that includes an imaging system configured to capture a view of at least a portion of a surgical site and to communicate the captured view, and a second medical device that includes one or more of a retracting device, an irrigation device, a suction device, a clipping device, a therapy delivery device, or a cutting device.

### CROSS-REFERENCE TO RELATED APPLICATIONS

The present application claims the benefit of and priority to U.S. Provisional Application No. 62/906,415, filed Sep. 26, 2019, which is hereby incorporated by reference herein in its entirety.

### FIELD OF THE TECHNOLOGY

The present disclosure relates generally to a miniaturized intra-body controllable medical device. More specifically, the present disclosure relates to the intra-body medical device having one or more systems for performing and/or assisting with aspects of a medical procedure.

### BACKGROUND

Many medical procedures require the physician to gain access to regions within the body in order to complete a diagnosis or provide a therapy to a patient. Often, physicians access internal regions of the body through the body's own natural orifices and lumens. Natural orifices include the nostrils, mouth, ear canals, nasolacrimal ducts, anus, urinary meatus, vagina, and nipples. The lumens include the interior of the gastrointestinal tract, the pathways of the bronchi in the lungs, the interior of the renal tubules and urinary collecting ducts, the pathways of the vagina, uterus, and fallopian tubes. From within these orifices and lumens, physicians can create an incision to gain access to almost any region of the body.

Traditional methods for gaining access to regions within the body include open surgical procedures, laparoscopic procedures and endoscopic procedures. Laparoscopic procedures allow the physician to use a small “key-hole” surgical opening and specially designed instruments to gain access to regions within the body. Initially, laparoscopic instruments were linear in nature, and required a straight obstruction free “line-of-sight” to access regions of the body. Endoscopic procedures allow the physician to access regions of the digestive system by passing flexible instruments through either the mouth or rectum.

Recently, physicians have begun to control these instruments using robots. These robots are typically connected in master/slave configuration, where the robot translates the physician's movements into instrument movements. Robotic controls have also allowed for advent of flexible laparoscopic instruments. Medical robots still require a physician to be actively controlling the movements and actions of the devices being controlled and require large expensive capital equipment and dedicated operating room spaces.

There are no means for providing this type of technology without the master/slave configuration, for example, controlling the motion of these devices, tracking or controlling the orientation, speed or location of these devices, accurately knowing where pictures were taken, and performing any type of surgical procedure or delivering therapy.

Thus, improvements are desirable in this field of technology. It would be beneficial to combine the ability to perform surgical procedures and provide therapy using robotic instruments with the footprint, size, and maneuverability of miniaturized systems or other structures. It would be beneficial to provide a means for controlling the movement of a medical device so that the surgeon can navigate it to a specific location.

### SUMMARY

There is disclosed herein a medical device for intra-body conveyance. In aspects, the medical device includes a host structure that has an interior area and can include one or more propulsion systems linked to the host structure. The host structure and the propulsion systems are configurable into a peripheral boundary of a size adapted to fit in a lumen or cavity of a living organism such as a human being or animal. The medical device can include one or more power supplies in communication with the propulsion systems. The medical device can include a control unit in communication with the propulsion systems and the power supplies. The control unit has a computer process controller configured to control the propulsion systems to move the host structure and the propulsion systems in the lumen so that the host structure and the propulsion systems are self-maneuverable within the lumen.

In aspects of the present disclosure, a system for assisting with a surgical procedure includes: a swarm of medical devices sized to be wholly deployed within a surgical site of a patient, where the swarm of medical devices is configured to operate concurrently within the patient to assist a surgeon perform a surgical procedure in the patient. The swarm of medical devices includes a first medical device that is sized to be wholly deployed within the surgical site and that includes an imaging system configured to capture a view of at least a portion of a surgical site and to communicate the captured view, and a second medical device that is sized to be wholly deployed within the surgical site and that includes at least one of: a retracting device, an irrigation device, a suction device, a clipping device, a therapy delivery device, or a cutting device.

In various aspects of the system, the swarm of medical devices includes a plurality of medical devices having imaging systems, where the plurality of medical devices includes the first medical device and is configured to cooperate to capture a 360-degree view of the surgical site.

In various aspects of the system, the second medical device includes the retracting device, and the swarm of medical devices includes a third medical device including a retracting device, where the second medical device and the third medical device operate to assist the surgeon perform the surgical procedure by concurrently retracting different portions of the surgical site using their respective retracting devices.

In various aspects of the system, the second medical device is configured to retract a gallbladder and the third medical device is configured to concurrently retract a liver.

In various aspects of the system, the second medical device includes at least one of a suction device or an irrigation device, and the second medical device is configured to assist the surgeon perform a dissection of Calot's triangle by creating an access window using at least one of: the suction device or the irrigation device.

In various aspects of the system, the second medical device is configured to create the access window behind cystic ducts.

In various aspects of the system, the first medical device is configured to assist the surgeon perform the surgical procedure by capturing a view of at least one of: a gallbladder, cystic artery, or cystic ducts.

In various aspects of the system, the second medical device includes the clipping device and is configured to assist the surgeon perform the surgical procedure by clipping a cystic artery.

In various aspects of the system, the swarm of medical devices includes a plurality of medical devices having clipping devices, where the plurality of medical devices includes the second medical device and is configured to cooperate to apply multiple clips to at least one of: the cystic artery or a cystic duct.

In various aspects of the system, the system includes a third medical device including a cutting device, where the third medical device is configured to cut at least one of the cystic artery or the cystic duct between two of the multiple clips.

In various aspects of the system, the swarm of medical devices includes a plurality of medical devices configured to cooperate to identify surgical complications in the surgical site.

In various aspects of the system, the plurality of medical devices is configured to cooperate to identify at least one of: bleeding or infection.

In various aspects of the system, the plurality of medical devices is configured to cooperate to identify at least one of: bile leak or bile duct damage that may require extensive bile duct reconstruction.

In various aspects of the system, at least one of the first medical device or the second medical device includes a pull device which includes a retractable anchor and a tether, where propulsion is generated by retracting the tether, thereby pulling at least one of the first medical device to the second medical device to a predetermined position.

In various aspects of the system, at least one of the first medical device or the second medical device includes a push device which includes a push rod adjacent to a fixed structure, wherein propulsion is generated by advancing the push rod, thereby pushing at least one of the first medical device or the second medical device to a predetermined position.

In various aspects of the system, at least one of the first medical device or the second medical device includes at least one of a pull device having magnets or a push device having magnets.

In various aspects of the system, at least one of the first medical device or the second medical device includes: an arrangement of inflating and deflating balloons, and a controller for controlling flow of a fluid to and from the balloons causing the balloons to expand and deflate, thereby creating propulsion of at least one of the first medical device or the second medical device.

In various aspects of the system, the first medical device includes a first power supply and the second medical device includes a second power supply, and the system includes a tether configured to connect the first power supply with the second power supply and configured to allow transfer of power between the first power supply and the second power supply through the tether.

In aspects of the present disclosure, a method for assisting with a surgical procedure includes: deploying a swarm of medical devices sized to be wholly deployed within a surgical site of a patient, where the swarm of medical devices includes: a first medical device that is sized to be wholly deployed within the surgical site and that includes an imaging system configured to capture a view of at least a portion of a surgical site and to communicate the captured view, and a second medical device that is sized to be wholly deployed within the surgical site and that includes at least one of: a retracting device, an irrigation device, a suction device, a clipping device, a therapy delivery device, or a cutting device. The method includes concurrently operating the swarm of medical devices within the patient to assist a surgeon perform a surgical procedure in the patient.

In various aspects of the method, the swarm of medical devices includes a plurality of medical devices having imaging systems, where the plurality of medical devices including the first medical device. The method includes operating the plurality of medical devices cooperatively to capture a 360-degree view of the surgical site.

In various aspects of the method, the second medical device includes the retracting device, and the swarm of medical devices includes a third medical device including a retracting device. The method includes operating the second medical device and the third medical device to assist the surgeon perform the surgical procedure by concurrently retracting different portions of the surgical site using their respective retracting devices.

In various aspects of the method, the second medical device includes at least one of a suction device or an irrigation device, and the method includes operating the second medical device to assist the surgeon perform a dissection of Calot's triangle by creating an access window using at least one of: the suction device or the irrigation device.

In various aspects of the method, the swarm of medical devices includes a plurality of medical devices having clipping devices, and the method includes cooperatively operating the plurality of medical devices to apply multiple clips to at least one of: the cystic artery or a cystic duct.

In various aspects of the method, the swarm of medical devices includes a third medical device having a cutting device, and the method includes operating the third medical device to cut at least one of the cystic artery or the cystic duct between two of the multiple clips.

In various aspects of the method, the method includes cooperatively operating the swarm of medical devices cooperate to identify surgical complications in the surgical site.

In various aspects of the method, at least one of the first medical device or the second medical device includes a pull device which includes a retractable anchor and a tether, and the method includes pulling at least one of the first medical device to the second medical device to a predetermined position by retracting the tether.

In various aspects of the method, at least one of the first medical device or the second medical device includes a push device which includes a push rod adjacent to a fixed structure, and the method includes pushing at least one of the first medical device or the second medical device to a predetermined position by advancing the push rod.

In various aspects of the method, at least one of the first medical device or the second medical device includes at least one of a pull device having magnets or a push device having magnets.

In various aspects of the method, at least one of the first medical device or the second medical device includes: an arrangement of inflating and deflating balloons, and a controller for controlling flow of a fluid to and from the balloons causing the balloons to expand and deflate, thereby creating propulsion of at least one of the first medical device or the second medical device.

In various aspects of the method, the first medical device includes a first power supply and the second medical device includes a second power supply, wherein a tether connects the first power supply with the second power supply, and the method includes allowing transfer of power between the first power supply and the second power supply through the tether.

### DESCRIPTION OF THE DRAWINGS

The drawings show aspects of the disclosed subject matter for the purpose of illustrating the disclosed technology. However, it should be understood that the present application is not limited to the precise arrangements and instrumentalities shown in the drawings, wherein:

FIG. 1A illustrates a representative intra-body controllable medical device formed in accordance with the present disclosure.

FIG. 1B illustrates a representative intra-body controllable medical device formed in accordance with the present disclosure.

FIG. 2 illustrates an alternative representation of an intra-body controllable medical device formed in accordance with the present disclosure.

FIG. 3 illustrates an intra-body controllable medical device featuring a helical screw drive propulsion system formed in accordance with the present disclosure.

FIG. 4 illustrates an intra-body controllable medical device featuring a sprocket driven track propulsion system formed in accordance with the present disclosure.

FIG. 5 illustrates an alternative representation of an intra-body controllable medical device featuring a sprocket driven track propulsion system formed in accordance with the present disclosure.

FIG. 6 illustrates an intra-body controllable medical device featuring a fluid/jet stream propulsion system formed in accordance with the present disclosure.

FIG. 7 illustrates an intra-body controllable medical device featuring a tentacle propulsion system formed in accordance with the present disclosure.

FIG. 8 illustrates an alternative representation of an intra-body controllable medical device featuring a tentacle propulsion system formed in accordance with the present disclosure.

FIGS. 9A, 9B and 9C illustrate an intra-body controllable medical device featuring an anchor and tether propulsion system formed in accordance with the present disclosure.

FIGS. 9D and 9E illustrate an intra-body controllable medical device featuring a push type propulsion system formed in accordance with the present disclosure.

FIGS. 9F and 9G illustrate an intra-body controllable medical device featuring magnetic field type propulsion system formed in accordance with the present disclosure.

FIG. 9H illustrates an intra-body controllable medical device with an anchor system and vacuum tube.

FIG. 9I illustrates an intra-body controllable medical device with a push rod system.

FIGS. 10A, 10B and 10C illustrate an intra-body controllable medical device featuring an inflating/deflating balloon propulsion system formed in accordance with the present disclosure.

FIG. 11 illustrates an intra-body controllable medical device featuring gyroscopic and wing/flipper stabilization systems formed in accordance with the present disclosure.

FIGS. 12A-G illustrate different scope systems for deploying an intra-body controllable medical device.

FIGS. 13A, 13B and 13C illustrate an intra-body controllable medical device being deployed by a scope.

FIGS. 14A and 14B illustrate an intra-body controllable medical device being deployed into the stomach by a scope.

FIGS. 15A and 15B illustrate systems for controlling an intra-body controllable medical device.

FIGS. 16A and 16B illustrate different power supply systems for powering an intra-body controllable medical device.

FIG. 17 illustrates the use of induction charging to power an intra-body controllable medical device.

FIG. 18 illustrates tethered power transfer between two intra-body controllable medical devices.

FIG. 19 illustrates intra-device storage systems for intra-body controllable medical devices.

FIGS. 20A and 20B illustrate imaging systems that can be incorporated with an intra-body controllable medical device.

FIGS. 21A and 21B illustrates the placement of a monitoring sensor by an intra-body controllable medical device.

FIGS. 22A, 22B and 22C illustrate the delivery of a therapy system by an intra-body controllable medical device.

FIGS. 23A-E illustrate different tissue and fluid sampling devices that may be used by an intra-body controllable medical device.

FIG. 24 illustrates material dispensing systems that may be used by an intra-body controllable medical device.

FIG. 25 illustrates an interactive group of intra-body medical devices.

FIG. 26 illustrates another interactive group of intra-body medical devices.

FIGS. 27A and 27B illustrate an exemplary operation that involves an interactive group of intra-body medical devices.

FIG. 28 illustrates a medical system according to aspects described herein.

FIG. 29 illustrates a flowchart of steps undertaken in an analysis of data obtained by an intra-body controllable medical device.

FIG. 30 illustrates an intra-body controllable medical device deployed in a patient and wirelessly connected to a processing device.

FIG. 31 illustrates images taken from an intra-body controllable medical device and compared to a data set of images.

### DETAILED DESCRIPTION

The present disclosure relates to an intra-body medical device having one or more of a propulsion system, a deployment system, a control system, a power supply system, an intra-device storage system, an imaging system, a therapy system, a sample and data gathering system, and/or a material dispensing system. Furthermore, in various aspects, the present disclosure details materials for an intra-body controllable medical device, an interactive group of intra-body controllable medical devices, configurations for intra-body controllable medical devices, and methods of using one or more intra-body controllable medical devices.

In various aspects, the present disclosure relates to miniaturized intra-body controllable medical devices. These may be externally controllable or may be fully autonomous. These may communicate via a tether or may communicate wirelessly. The intra-body medical device may have a propulsion system, a deployment system, a control system, a power supply system, an intra-device storage system, an imaging system, a therapy system, a sample and data gathering system, and/or a material dispensing system. Any of the medical devices described herein may work independently or work together in a group. The interactive group of medical devices can include two or more medical devices, such as three medical devices, four medical devices, five medical devices, six medical devices, or more than six medical devices. Aspects of the medical devices are described in International Patent Application Publication No. WO2019/191207, filed on Mar. 27, 2019, and is incorporated by reference herein in its entirety.

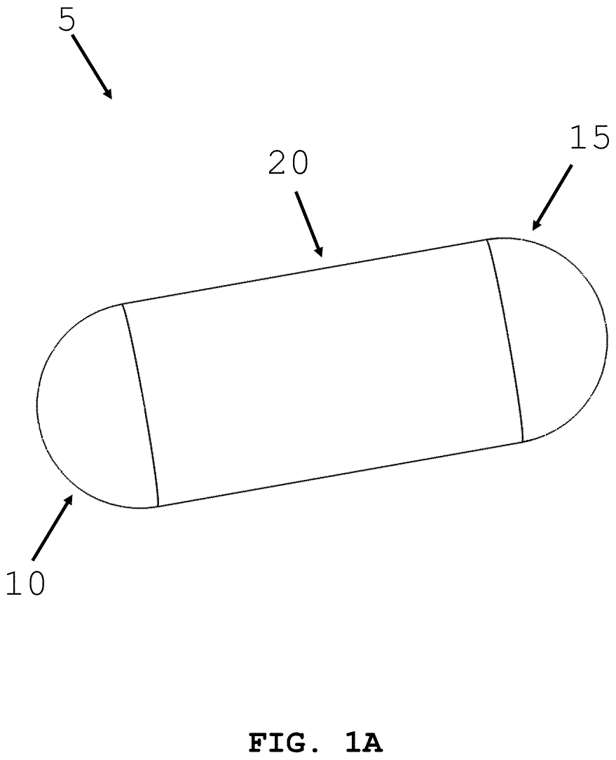

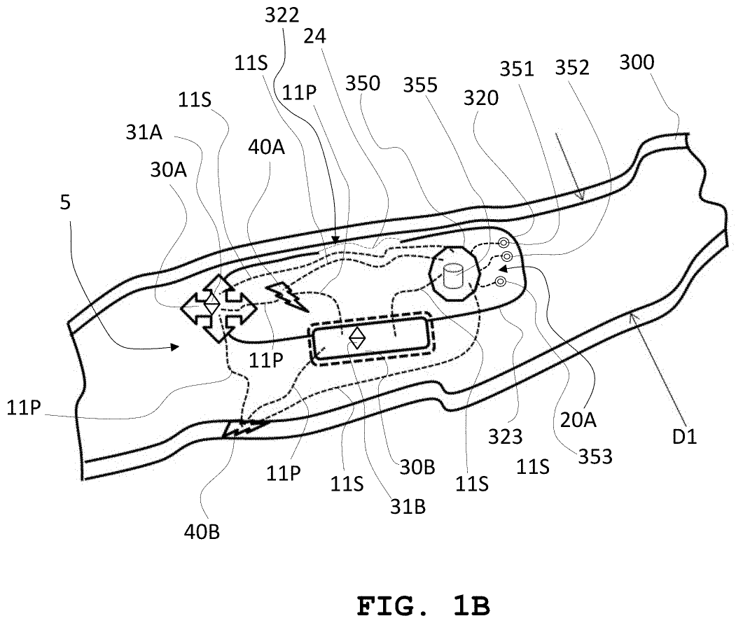

FIG. 1A illustrates an exemplary intra-body controllable medical device (also referred to herein as “a medical device”). In some aspects, the intra-body controllable medical device 5 is spherocylindrical. The intra-body controllable medical device may be shaped according to the anatomy that it will need to navigate, the method used to deliver it, and/or the actions the medical device needs to perform. Intra-body controllable medical device 5 has a distal end 10, a proximal end 15, and body 20 connecting the distal end 10 and proximal end 15. A control unit, a power supply system, an intra-device storage system, an imaging system, a therapy system, a sample and data gathering system, and a material dispensing system may be located within body 20 of the medical device 5, as described herein. The intra-body controllable medical device may be sized according to the anatomy that it will need to navigate, the method used to deliver it and/or the actions the medical device needs to perform. As an example, overall dimensions for an intra-body controllable device operating within the gastrointestinal track may have a diameter of about 25 mm and a length of about 75 mm. In some aspects, the device may have a diameter of about 15 mm and a length of about 50 mm. In some aspects, the diameter may be less than about 15 mm and a length of less than about 50 mm. Overall dimensions for an intra-body controllable device that is delivered using a scope may have a diameter of about 20 mm in diameter and a length of about 75 mm. In some aspects, the diameter may be about 15 mm and the length may be about 50 mm. In some aspects, the diameter may be less than 15 mm and the length less than 50 mm. Control system, power supply system, intra-device storage system, imaging system, therapy system, sample and data gathering system, and material dispensing systems are sized to fit within these dimensional guidelines.

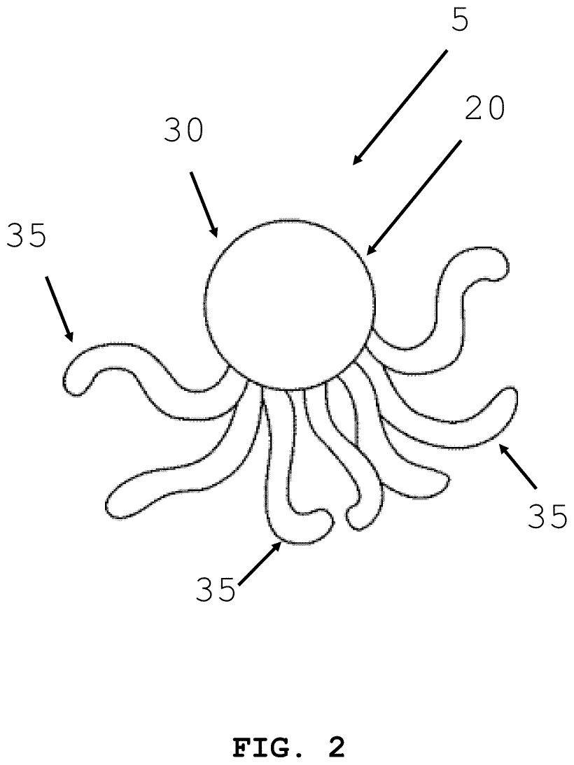

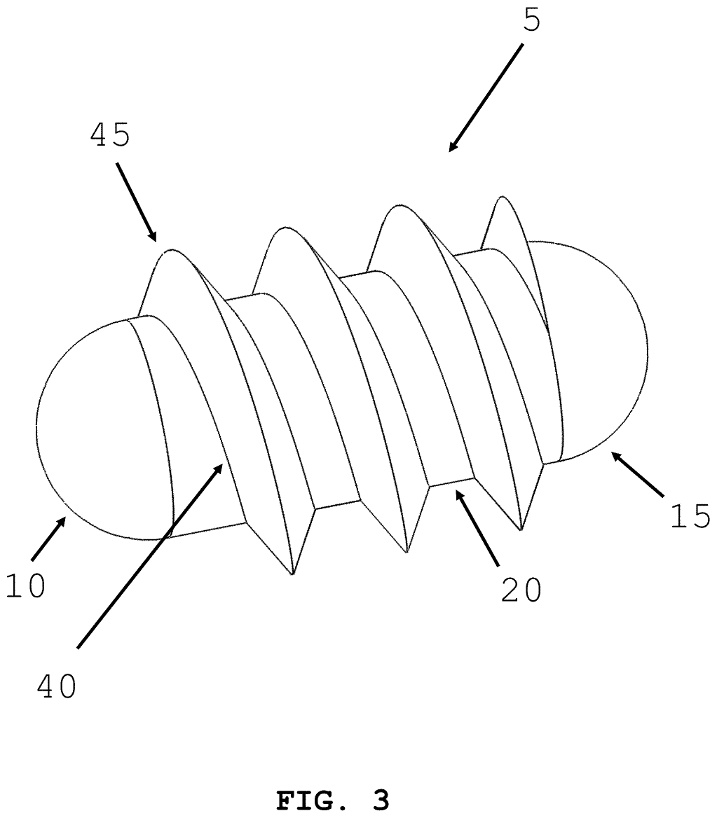

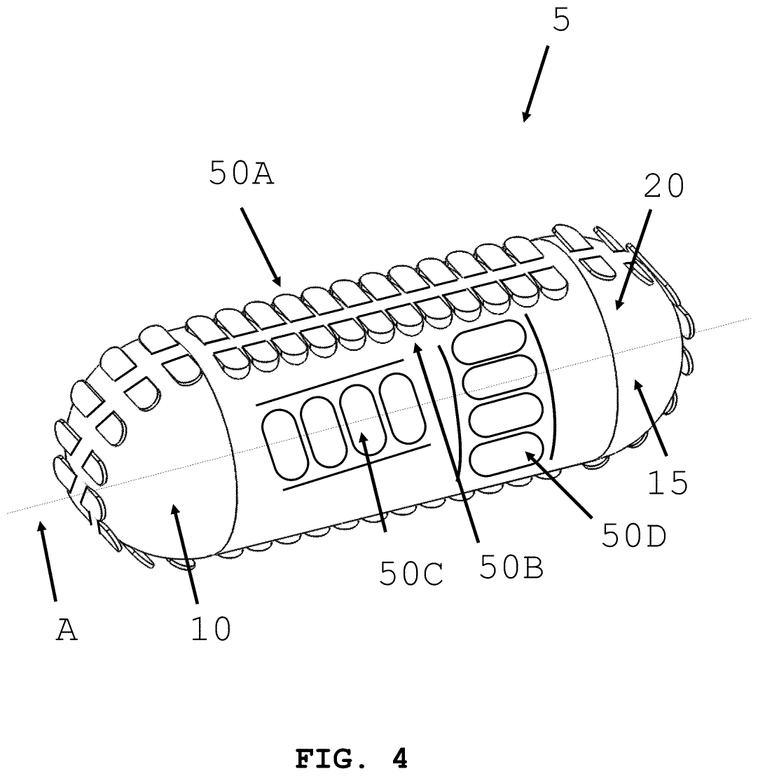

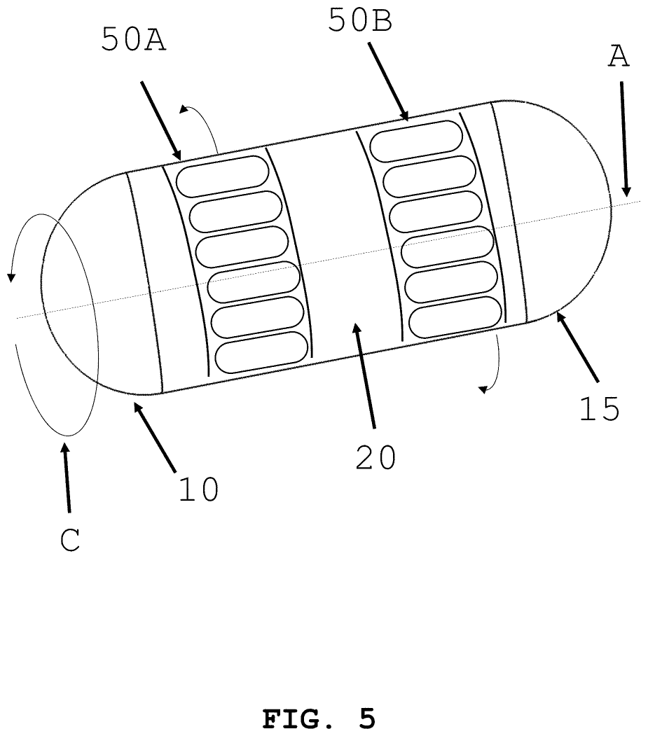

As shown in FIG. 1B, the medical device 5 includes the body 20 which is a host structure 320 that has an interior area 20A. A first propulsion system 30A and a second propulsion system 30B (e.g., a sprocket and track system similar to those shown and described with reference to FIGS. 4 and 5) are linked to the host structure 320. While the first propulsion system 30A and a second propulsion system 30B are shown and described, the present disclosure is not limited in this regard as only one propulsion system or more than two propulsion systems may be employed without departing from the broader aspects of the present disclosure. The first propulsion system 30A (e.g. see FIGS. 2-11) and the second propulsion system 30B are configurable into a peripheral boundary 323 (e.g., a skin or exterior surface) of a miniaturized size and are adapted to fit in a lumen 300 (or tissue, muscle or fat) of a living organism, such as a human, or larger cavities such as the abdominal cavity, pelvic cavity, thoracic cavity and/or the dorsal body cavity. In some aspects, the medical device 5 is configured to navigate in bone marrow within a bone. As an example, overall dimensions for an intra-body controllable device operating within the gastrointestinal track may have a diameter of about 25 mm and a length of about 75 mm. In some aspects, the device may have a diameter of about 15 mm and a length of about 50 mm. In some aspects, the diameter may be less than about 15 mm and a length of less than about 50 mm. Overall dimensions for an intra-body controllable device that is delivered using a scope may have a diameter of about 20 mm in diameter and a length of about 75 mm. In some aspects, the diameter may be about 15 mm and the length may be about 50 mm. In some aspects, the diameter may be less than 15 mm and the length less than 50 mm. In some aspects, the host structure 320 includes an opening 322 therein for access to the interior area 20A of the host structure 320. In some aspects, a retractable, removable or pivotable member 24 (e.g., a door, window or flap) selectively covers the opening 322. Propulsion systems 30A and 30B may be used to move device 5 within lumen 300. Additionally, one or more propulsion systems 30A and 30B may include one or more orientation control devices 31A and 31B such that the propulsion systems can generate smaller and or finer movements to maintain the position of the device within the lumen 300 and can be used to change the orientation of the device within the lumen 300, tissue, muscle or fat. Controlling the orientation of the medical device 5 within the lumen 300, tissue, muscle or fat allows the intra-device storage system, imaging system, therapy system, sample and data gathering system, and/or a material dispensing system to be adjacent to a region of interest within the lumen, tissue, muscle, bone marrow or fat.

As shown in FIG. 1B, a first power supply 40A and a second power supply 40B are in communication (e.g., via power supply conductors or transmission lines or channels generally designated by the dashed lines marked 11P) with the first propulsion system 30A and the second propulsion system 30B. While the first power supply 40A and the second power supply 40B are shown and described as being in communication with the first propulsion system 30A and the second propulsion system 30B, the present disclosure is not limited in this regard as only one power supply or more than two power supplies may be employed and any of the power supplies (e.g., 30A or 30B) may be in communication with one or more propulsion systems (e.g., 40A or 40B).

As shown in FIG. 1B, a control unit 350 is in communication (e.g., via signal transmitting lines, wires or wireless channels, generally designated by dashed lines marked 11S) with the first propulsion system 30A, the second propulsion system 30B, the first power supply 40A and the second power supply 40B. The control unit 350 includes a computer process controller 355 that is configured to control the first propulsion system 30A, the second propulsion system 30B to move the host structure 320, the first propulsion system 30A and the second propulsion system 30B in the lumen 300 so that the host structure 320, the first propulsion system 30A, the second propulsion system 30B and the control unit 350 are self-maneuverable within the lumen 300.

As shown in FIG. 1B, a tracking device 351, a signal transmitter 352 and a signal receiver 353 are in communication with the control unit 350 via signal lines 11S for tracking and guiding the medical device 5 within the lumen 300.

As shown in the exemplary embodiment of FIG. 2, the intra-body controllable medical device 5 may be octopus shaped. The intra-body controllable medical device has a main body 30, and appendages 35. Appendages 35 may be used for propulsion, covering or wrapping the host structure 20, forming a portion of the host structure 20 or to perform a therapeutic or diagnostic task. A control unit, power supply systems, an intra-device storage system, an imaging system, a therapy system, a sample and data gathering system, and a material dispensing system similar to those shown and described with reference to FIG. 1B, may be located within main body 30 and/or appendages 35 of the device or in the interior areas of the host structure 20.

As shown in FIGS. 3-11, the present disclosure includes an intra-body controllable medical device and more particularly to propulsion systems for moving the intra-body controllable medical device within a lumen or orifice. The propulsion systems include one or more orientation control devices 31A, 31B per FIG. 1B, for controlling the orientation of the device within the lumen or orifice. The intra-body controllable medical device is sized to travel through lumens, cavities and/or orifices tethered or untethered. Thus, the intra-body controllable medical device is equipped with one or more propulsions systems, including but not limited to:

(1) a sprocket driven track structure in communication with the device; (2) fluid jet stream discharging from the device; (3) an arrangement of inflating and deflating balloons in predetermined positions on and/or around the device; (4) a plurality of articulating tentacles extending from the device; (5) a screw-drive formed on external surfaces of the device and (6) stabilization wings, flippers, anchors, braces, supports and/or clamps, as described herein. The intra-body controllable medical device may also move within the body through peristalsis of the digestive system. In some aspects, a propulsion system may be used to move device 5 to a region of interest. The device may then exit the body passively through peristalsis or may be withdrawn from the body by a device such as a tether.

Referring to FIG. 3, an intra-body controllable medical device with a screw-drive propulsion system is shown. The screw drive propulsion system has a helical thread on the external surface of the device. The helix 40 circumscribes the body 20 of the intra-body controllable medical device. A screw thread 45 is swept around the helix 40. Rotation of the helix 40 around the central axis of body 20 causes the intra-body controllable medical device to advance in a lumen or orifice such as lumen 300 of FIG. 1B. Switching the direction of rotation of the helix 40 causes the intra-body controllable medical device to advance in the opposite direction.

In an alternative aspect and referring to FIG. 4 and FIG. 5, an intra-body controllable device with a sprocket driven track structure in communication with the device is shown. The track 50A-D may be oriented either along the axis A of the body 20 (FIG. 4), circumferentially around body 20 (see arrow C in FIG. 5) or along one or more portions of the host structure 20 (see the second propulsion system 30B in FIG. 1B). A sprocket (not shown) may be housed within the proximal end 10 and distal end 15 of the device (FIG. 4) or central to the body 20 (FIG. 5). Movement of the track 50 relative to the body 20 of the intra-body controllable medical device 5 generates motion of the medical device.

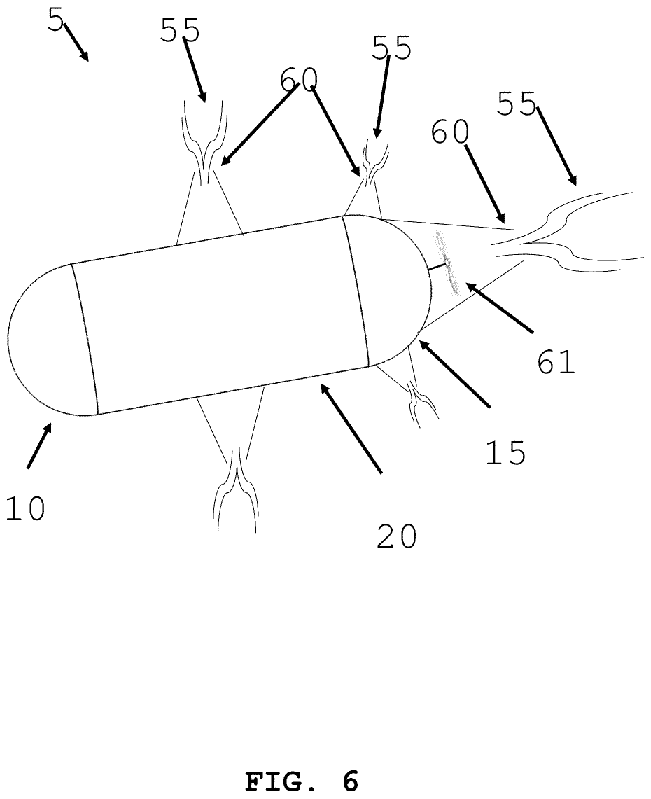

In an alternative aspect and referring to FIG. 6, an intra-body controllable medical device with one or more fluid/gas jet stream discharge propulsion systems is shown. The jet stream 55 matter (e.g., gas, liquid, gel, or particles) may be released from intra-body controllable medical device 5 through a nozzle or orifice 60. The orifice 60 may be located on the distal end 10 and/or the proximal end 15 of the device. The jet stream 55 matter may be stored within body 20 of the device. Alternatively, the matter may be harvested from the body (e.g. gastricjuice). Alternatively, the fluid may be harvested from the body (e.g. gastric juice) and reacted with a compound stored within device 20 (e.g. sodium bicarbonate) to create a gas (e.g. carbon dioxide gas) which can be released as a fluid/gas jet stream 55 under pressure through the orifice 60 to create propulsion. Additionally, a propeller and or turbine 61 may be located within nozzle or orifice 60. The jet stream of matter 55 may turn the turbine to generate thrust. Additionally, fluid/gas jet stream discharge propulsion system may be used as an orientation control system 31A and 31B.

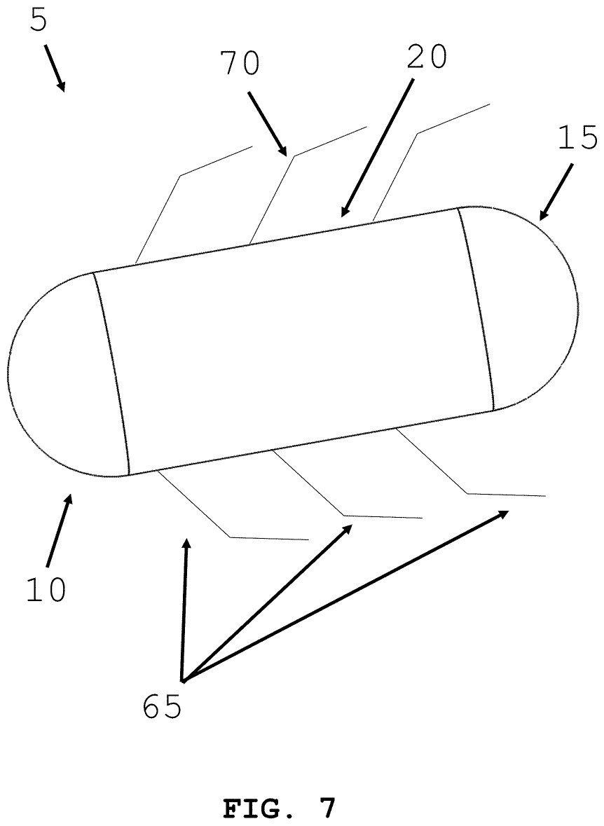

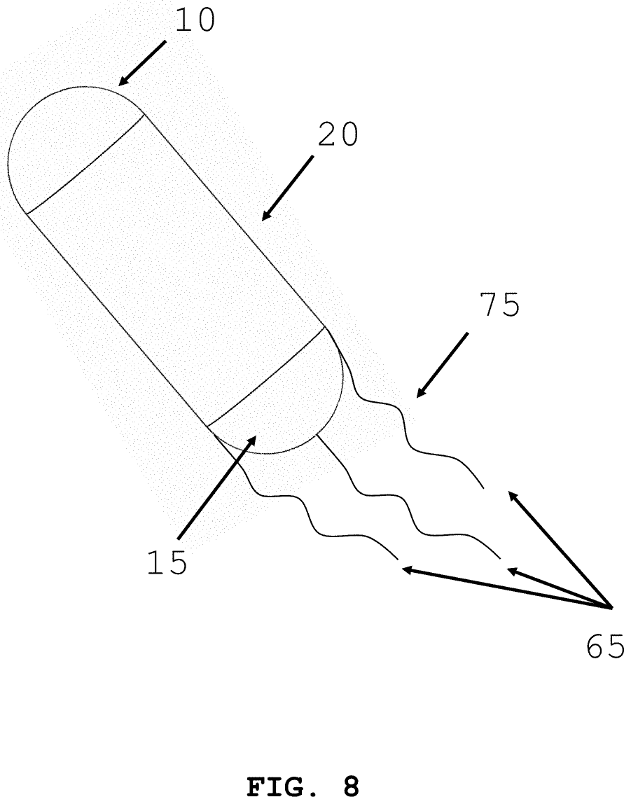

In an alternative aspect and referring to FIG. 7 and FIG. 8, an intra-body controllable medical device 5 with a plurality of articulating tentacles 65 extending from the body is shown. A plurality of tentacles 65 may be disposed along the length of the body 20 of the device (FIG. 7); alternatively, the tentacles 65 may be located on the distal end 10 or proximal end 15 of the device (FIG. 8). The tentacles 65 may be linear. The tentacles 65 may be linear with hinged regions 70 or may be able to articulate throughout their length 75. Motion of the tentacles 65 generates propulsion of the intra-body controllable medical device.

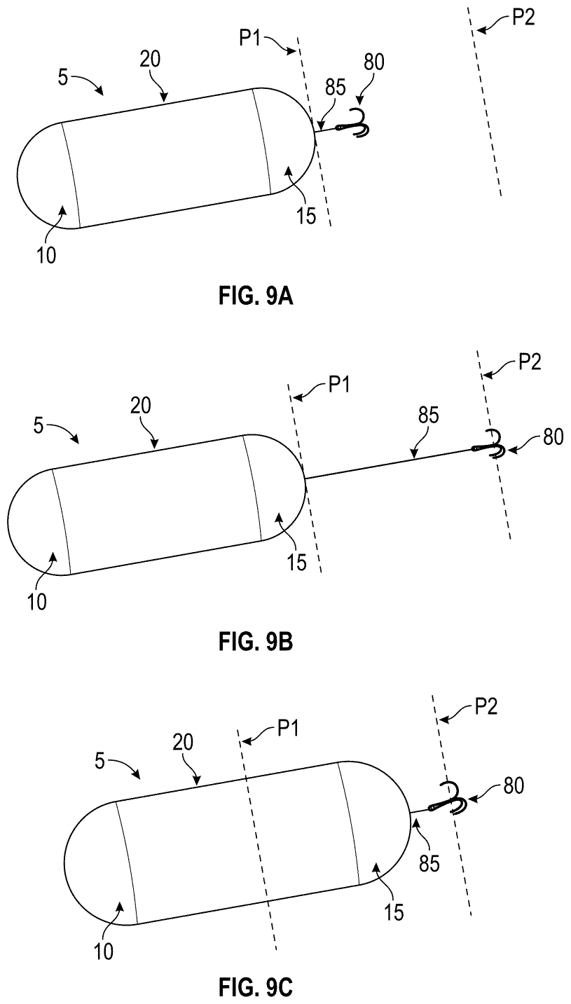

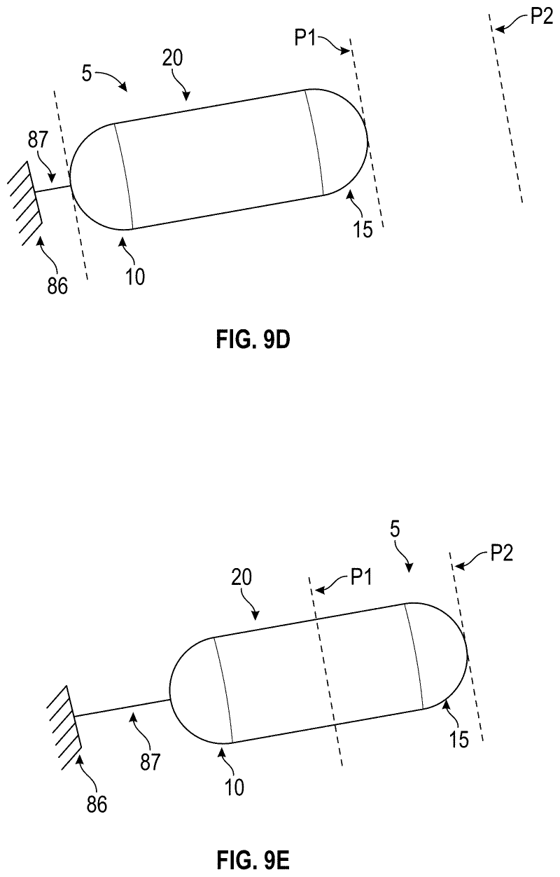

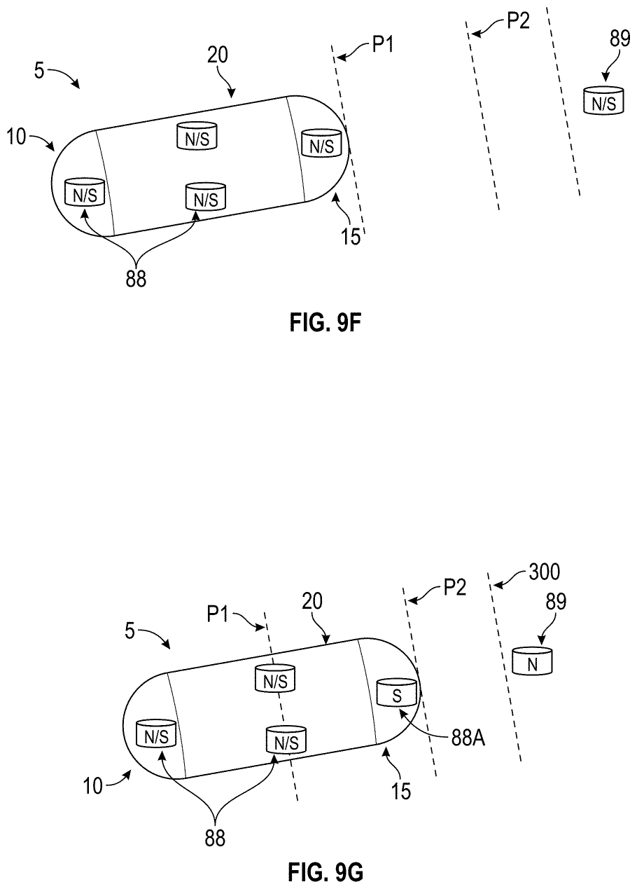

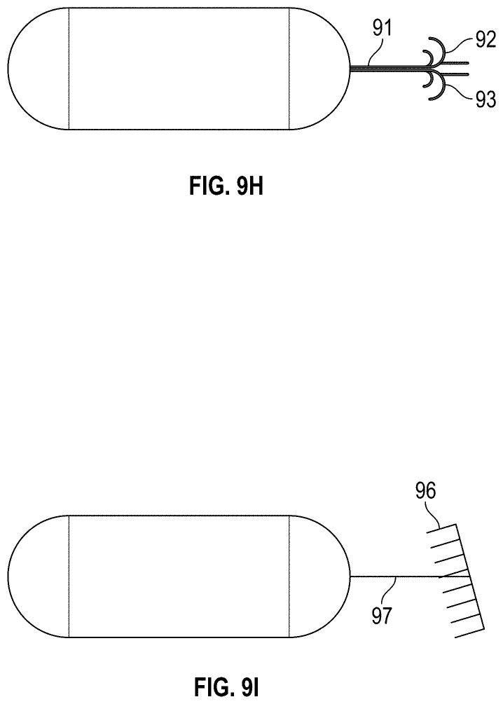

In an alternative aspect and referring to FIGS. 9A-9G, an intra-body controllable medical device 5 with a push or pull propulsion system is shown. As shown in FIGS. 9A, 9B, and 9C, a retractable anchor-based propulsion system is shown. An anchor 80 can be any kind of anchor known in the art. As shown in FIG. 9A, the proximal end 15 is at position P1 and the anchor 80 is in the retracted position. As shown in FIG. 9B, the anchor 80 is deployed via an extended tether 85 and attaches to tissue at position P2. The anchor 80 is connected to the intra-body controllable medical device 5 by a tether 85. Propulsion is generated by retracting the tether 85 (FIG. 9C), thereby pulling the medical devices to the position P2. In some aspects, where an interactive group of medical device is used, a first medical device can deploy the anchor 80 via an extended tether 85 and attach to a second medical device, e.g., via a loop (not shown), on the second medical device, thereby pulling the medical device to an advanced position.

In an alternative aspect and referring to FIGS. 9D and 9E a push propulsion system is shown. As shown in FIG. 9D the proximal end 15 is at position P1 and push rod 87 is in the retracted position. The end of push rod 87 may be adjacent to a fixed structure 86. Fixed structure 86 may be lumen 300, a probe, or a scope. Propulsion is generated by advancing push rod 87 (FIG. 9E) thereby pushing the medical device to the position P2. In some aspects, where an interactive group of medical device is used, a first medical device can push against a surface of a second medical device to an advanced position.

In an alternative aspect and referring to FIG. 9F and FIG. 9G a push and or pull propulsion system is shown. As shown in FIG. 9F, push and or pull propulsion system utilizes magnets or magnetic fields to move device 5. Magnets may be permanent or electromagnetic. Magnets 88 are located within device 5. Additionally, there may be one or more magnets 89 located outside of lumen 300. Magnets 88 and 89 are configured to have either a north pole or a south pole. Magnets 89 may be located outside of the organism. Proximal end 15 is located at position P1. Propulsion is generated by creating an attraction force between magnet 89 and magnets 88 (FIG. 9G). An attractive force is generated between magnet 88A's south pole and magnet 89's north pole. This attractive force moves the medical device to position P2. Alternatively, magnet 88A's south pole (or north pole) may be aligned with magnet 89's south poll (or north pole), a repulsive force can be generated and used to push medical device 5. In some aspects, where an interactive group of medical device is used, a first medical device can include at least one magnet, and a second medical device can include at least one magnet where a magnet in the first medical device south pole (or north pole) may be aligned with a magnet in the second medical device south poll (or north pole), and a repulsive force can be generated and used to push medical device 5.

As shown in FIG. 9H, a drone with an anchor system 91 can deploy a vacuum tube 92 to remove material from lumen, such as plaque. In various aspects, the drone can be used in the gastrointestinal tract to remove obstruction using the illustrated anchor and vacuum deployment system. In various aspects, the tube 92 can be used to deposit material 93, such as material that can dissolve plaque or gastrointestinal obstruction prior to vacuum of debris.

As shown in FIG. 9I, a drone with a push rod system 96 can be used for tissue manipulation during surgery to improve access to surgical site. The drone can be used to push tissue or fixed structures by gripping and pulling. In various aspects, the drone has six degrees of freedom 97 around the push rod allowing it to create the angles required for pushing and gripping/pulling maneuvers.

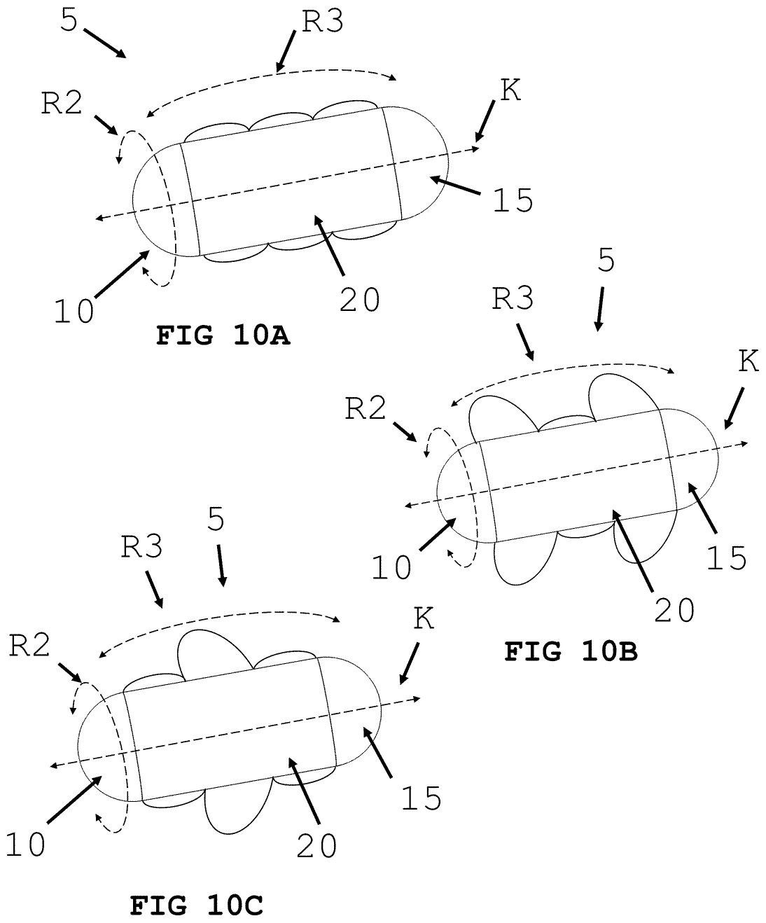

In an alternative aspect and referring to FIG. 10, an intra-body controllable medical device 5 with an arrangement of inflating and deflating balloons 90 in predetermined positions in the direction of the arrows R and orientations (e.g., rotational or angular movement as indicated by the arrows R2 and R3) on and/or around the device is shown. The balloon 90 may be made of an elastomeric material that can be expanded under pressure yet return to its original configuration when the pressure is released. The balloon 90 may be filled with a fluid and/or a gas. When the balloon 90 is filled, the balloon increases in volume and changes shape. As an example, the balloon 90 may change to shape conformation 95 when filled with a fluid and/or gas. The fluid and/or gas may be stored within the body 20 of the medical device 5. Alternatively, the fluid may be harvested from the body (e.g. gastric juice). Alternatively, a fluid may be harvested from the body (e.g. gastric juice) and reacted with a compound stored within the device (e.g. sodium bicarbonate) to create a gas (e.g. carbon dioxide). This gas can then be used to fill and expand the balloon 90. A controller can be located within the device to direct the fluid and/or gas flow to different balloons. The rhythmic expansion and contraction of balloons can create propulsion.

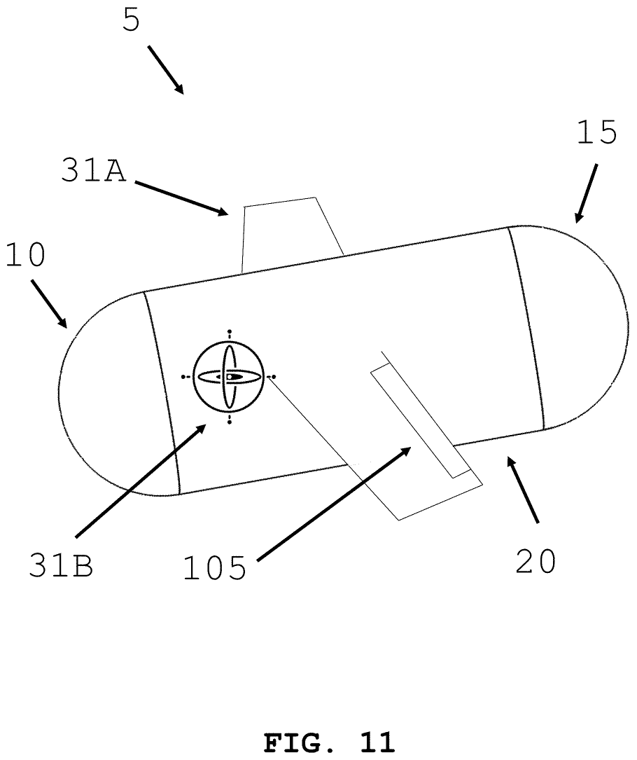

In an alternative aspect and referring to FIG. 1B and 11, an intra-body controllable medical device 5 may be equipped with an orientation control device (e.g. stabilization wing 31A, 31B). The orientation control device (e.g. stabilization wing 31A, 31B) is compatible with any of the propulsion systems disclosed herein. The orientation control device (e.g. stabilization wing 31A, 31B) can help guide the movement of the medical device 5 within the lumen. The orientation control device (e.g. stabilization wing 31A, 31B) may further have a flap 105 to further provide stabilization and guidance. Orientation control device may also be a gyroscope 31B. Gyroscope 31B may be used to provide stability or maintain a reference direction.

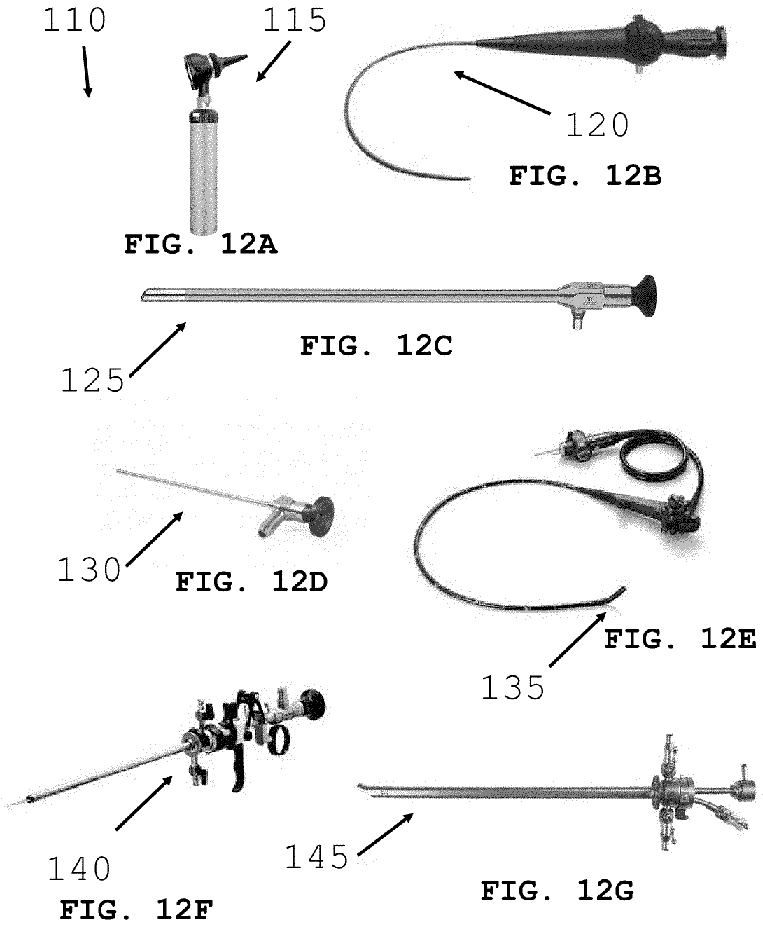

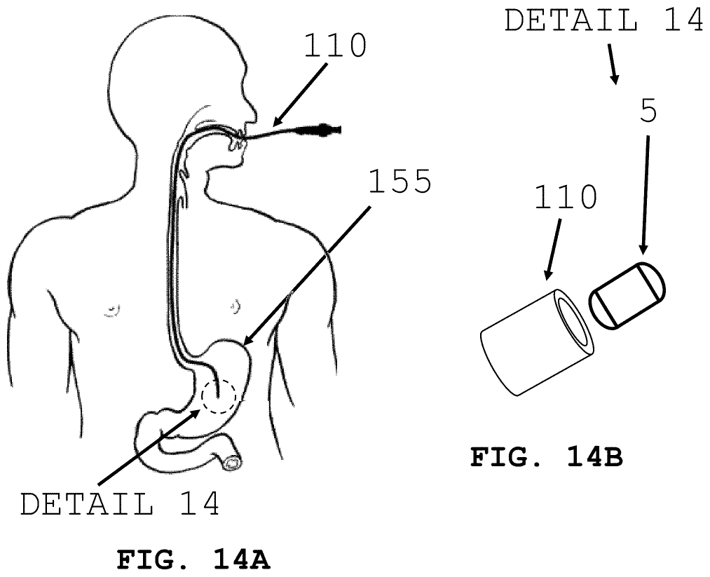

As shown in FIGS.12A-G through FIG. 14, the present disclosure includes an intra-body controllable medical device 5 and more particularly to deployment devices and methods for deploying an intra-body medical device into the lumen 100. In particular, scopes for medical applications having rigid shafts or flexible conduits are configured with one or more device storage compartments, channels, actuation devices, tethers and discharge ports for deployment from one or more portions of the probe portion of the scope while positioned in a lumen. Referring to FIG. 12, the deployment device is configured to be integrated with various medical scopes 110 including but not limited to an ENT otoscope 115, a naso-pharyngoscope 120, a laparoscope 125, a sinuscope 130, a colposcope 135, a resectoscope 145 and a cystoscope 150. Furthermore, medical device 5 may be deployed through a tube instead of a scope. Additionally, medical device 5 may be deployed via a catheter into a blood vessel or may be surgically placed (e.g. after heart surgery). Medical device 5 may be deployed though an appropriately sized needle (e.g. to gain access bone marrow) and may also be deployed generally to any area within the body (e.g. muscle, fat, and tissue) or on the skin. Medical device 5 may be deployed on the skin at the site of a wound and provide therapy (e.g. discharge clotting material like zeolite or antibacterial medication).

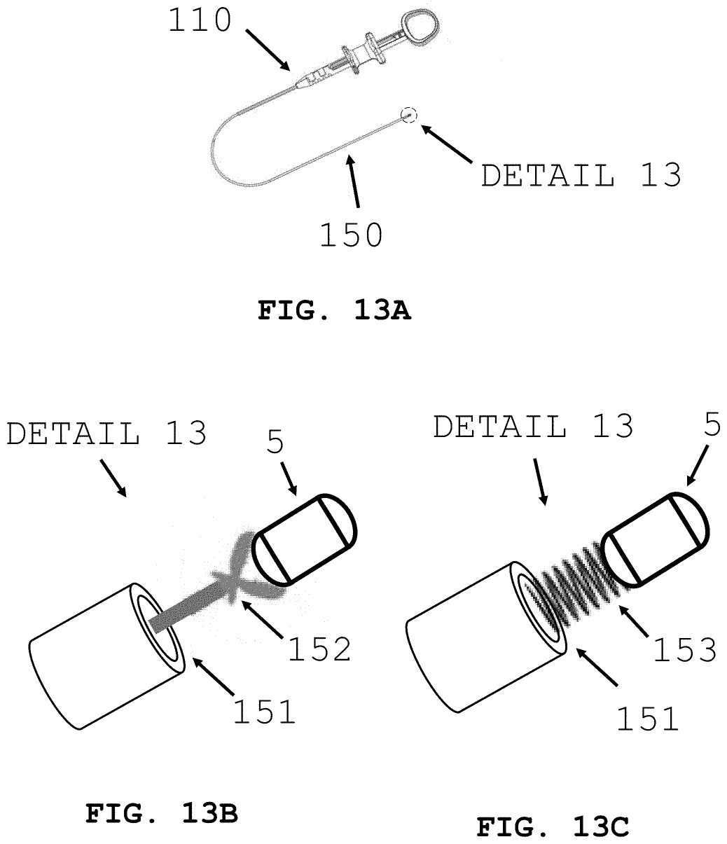

As shown in FIG. 13A, the intra-body controllable medical device 5 can be deployed through the working channel 150 of the endoscope 110. The end of the working channel 150 may have a docking station 151 (FIG. 13B and FIG. 13C.) Docking station 151 may utilize a claw 152 (FIG. 13B) or a spring 153 (FIG. 13C) to hold and deploy medical device 5. Furthermore, and referring to FIG. 14A and FIG. 14B, this method for deployment of the intra-body controllable medical device in the lumen 100 further includes the use of a scope 110 to deliver the device directly to the stomach 155. Alternatively, the scope 110 may be used to deliver the device directly to various organs, for example, the bladder. The method for deployment of the intra-body medical device in a lumen further includes digestion through the oral cavity, inhalation of one or more nano-sized versions of such devices for introduction to the respiratory system of a human, including the nose, pharynx, larynx, trachea, bronchi and lungs.

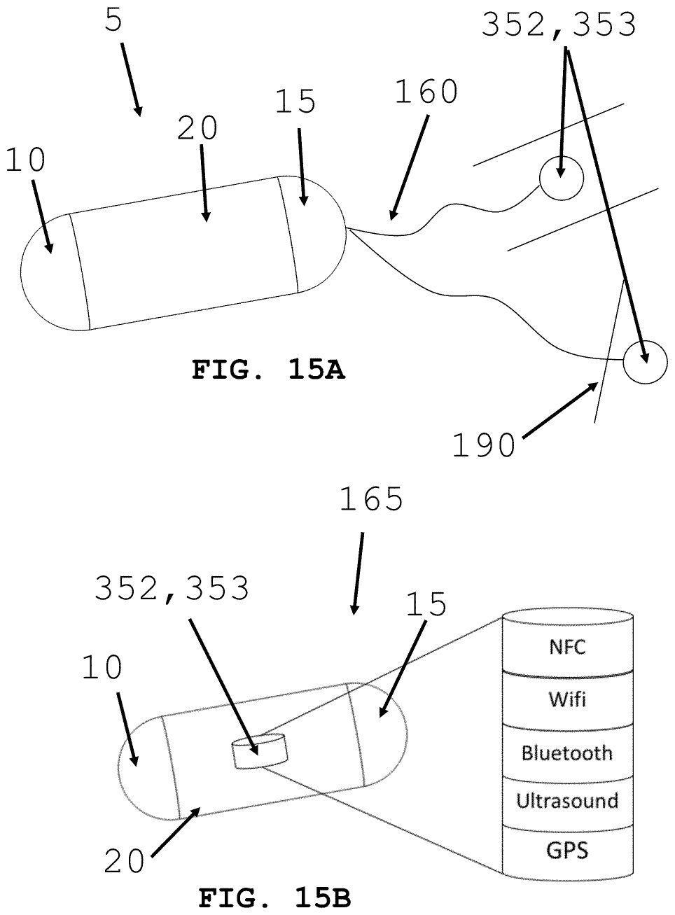

As shown in FIG. 15A and FIG. 15B, the present disclosure includes an intra-body controllable medical device 5 and more particularly to control and communications systems and methods for controlling and communicating with the intra-body controllable medical device in a lumen. In particular, the control and communications systems are configured to identify and track the location and orientation of the device relative to predetermined locations in the lumen and to control the device propulsion and orientation systems to guide the device to, from and around the predetermined position.

As shown in FIGS.1B and 15A and B, the control unit 350 includes hard wired 160 (FIG. 15A) and/or wireless 165 (FIG. 15B) communication devices (e.g., transmitters 352 and receivers 353) linking an external command and monitor center with a computerized process controller 55 (FIG. 1B) in the medical device 10 which is in communication with and controls the operation of the propulsion and orientation systems based upon real time position information of the device in the body. The control unit 350 includes a software algorithm on a computer readable medium that is operable with the computerized process controller to effectuate the identification, tracking and control of the intra-body controllable medical device within a lumen.

The control unit 350 includes tracking devices 351, transmitters 352 and receivers 353, see FIG. 1B and FIG. 15 including GPS, radiation emitting sources/radiation monitoring devices, ultra sound devices, near field communication devices, cellular data (2G/3G/4G/5G) devices, Wi-Fi devices, and Bluetooth devices, that are configured to determine the position of the intra-body controllable medical device in the lumen, similar to those shown and described with reference to element numbers 315, 352 and 353 in FIG. 1B.

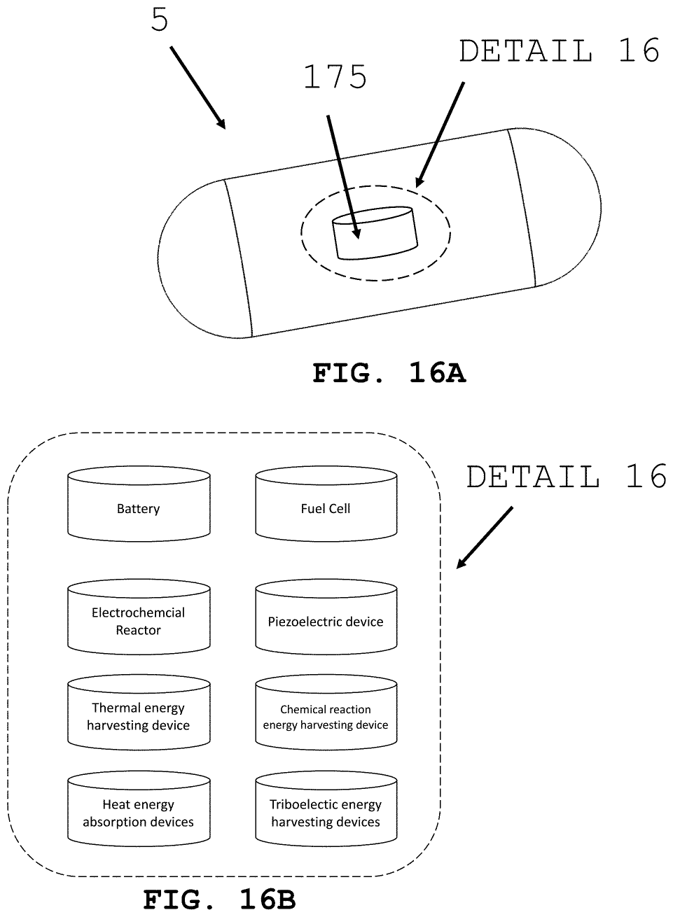

As shown in FIG. 16A and FIG. 16B, the present disclosure includes power supply systems 175 for an intra-body medical device and more particularly to miniaturized (e.g., computer chips having integrated circuits and positioned on integrated circuit boards). The power supplies and storage devices that provide power for propulsion, control and operation of subcomponents within and around the intra-body controllable medical device and ancillary devices connectable to the intra-body controllable medical device can be miniaturized. In particular, the miniaturized power supplies can include batteries, fuel cells, electrochemical reactors, piezoelectric devices, energy harvesting devices that obtain thermal and/or chemical reaction energy from the fluids in and tissue of the lumen and adjacent organs, thermal reactors heat absorption energy conversion devices and triboelectric energy harvesting devices. Batteries may include any of the kind known in the art including, but not limited to, alkaline batteries, atomic batteries, lead-acid batteries, lithium ion batteries, magnesium-ion batteries, nickel-cadmium batteries, nickel metal hydride batteries and rechargeable alkaline batteries. Electrochemical reactors may store the chemical required to create electricity within the device. Alternatively, electrochemical reactors may use fluids found within the body to react with chemicals stored within or on the device to create electricity. Piezoelectric devices may create electricity by harvesting either the body's own motion (e.g. peristalsis) or the motion of the device as it moves within the lumen. Heat absorption devices may harvest energy from the body's temperature to create electricity. Triboelectric energy harvesting devices generate electricity as the body of the device comes into frictional contact with the lumen of the body it is passing within. Additionally, energy may be stored by the device using capacitors, thermal medium, batteries and mechanical expansion devices (e.g., springs and balloons).

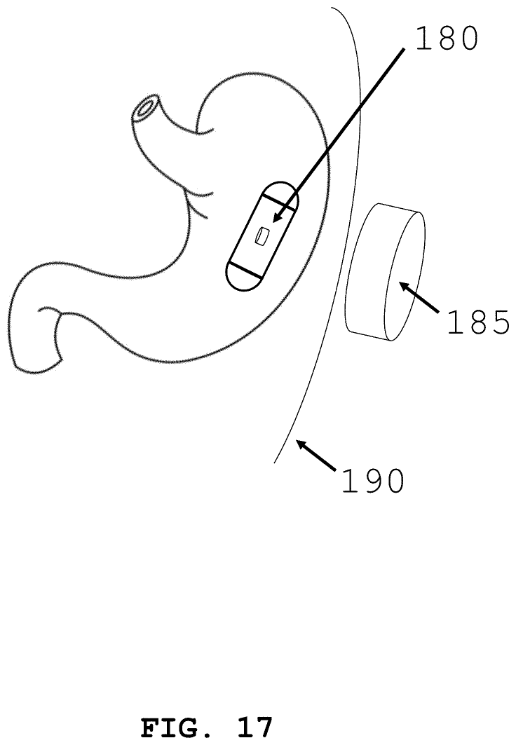

Additionally, as seen in FIG. 17, the intra-body controllable medical device 5 can be directly powered by induction energy transfer from the outside of the body 190 or inside of the body 190. An induction energy receiver 180 can be located within device 5. An induction energy transmitter 185 can be located outside body 190. Alternatively, the device may function on another internal energy storage device and be recharged by induction, charging when sufficient stored electricity has been consumed.

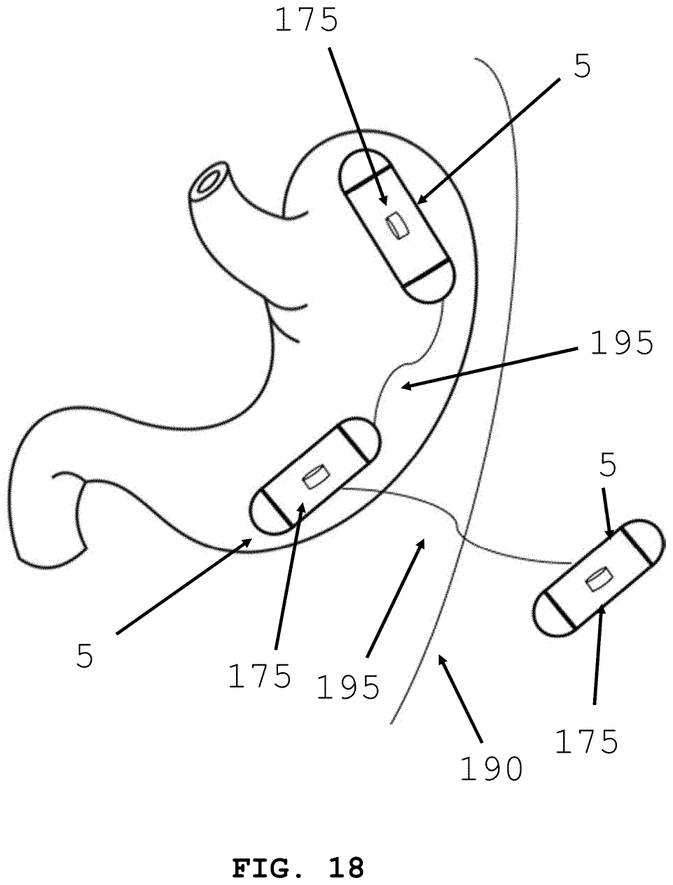

Alternatively, as seen in FIG. 18, one intra-body controllable medical device 5 can be tethered to a second intra-body controllable medical device 5. Tether 195 can transfer electricity from power source 175 in a first device to a second power source 175 of the second device. The second intra-body controllable medical device 5 may be located inside the body 190 or outside the body 190. The two devices may be permanently tethered together, or they may tether when the transfer of electricity is required.

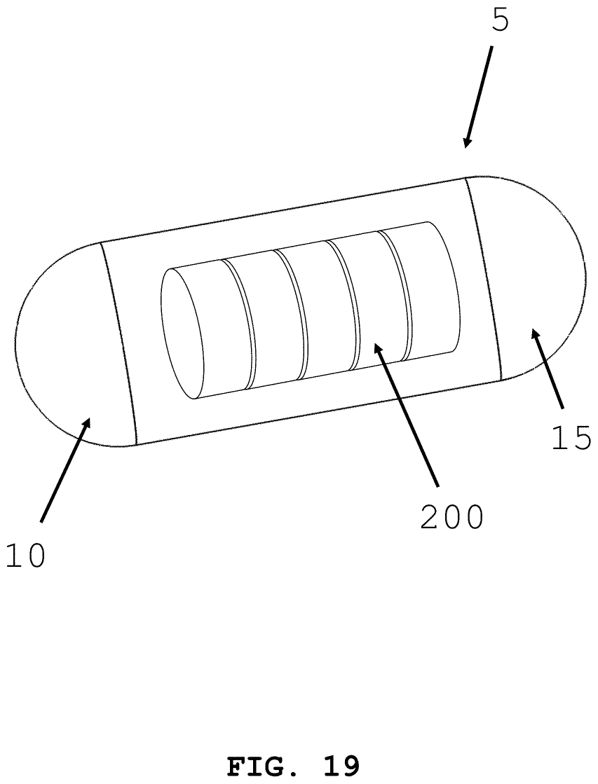

As shown in FIG. 19, the present disclosure includes an intra-body medical device having intra-device storage systems 200 therein and more particularly to miniaturized compartments for housing one or more power supplies, energy storage devices, medications, imaging systems, computer processor controllers, communications transmitters and receivers, propulsion systems, therapy delivering devices (e.g., radiation sources), process waste, biopsies, blood and tissue samples, medical and surgical instruments, fluids, gases, powders and consumables. The storage compartments are configured with walls, internal and external support structures, inlets, outlets, sensors (e.g., temperature, pressure and chemistry sensors), valves, pumps and ingress/egress apertures. The intra-device storage system 200 may be used to hold nerve blocking and stimulating drugs and devices, may hold devices for cleaning plaque from artery walls or may hold and deploy intestinal restrictive bands.

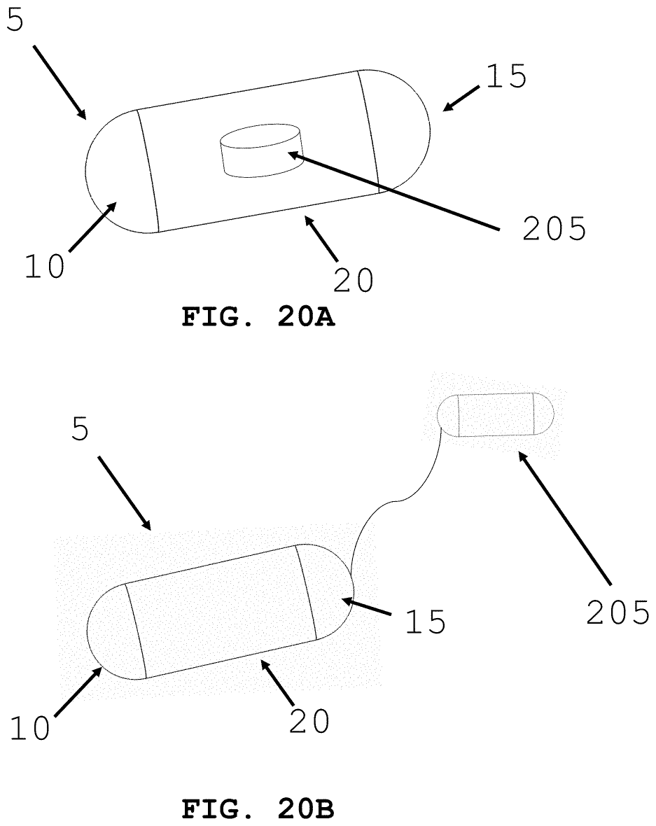

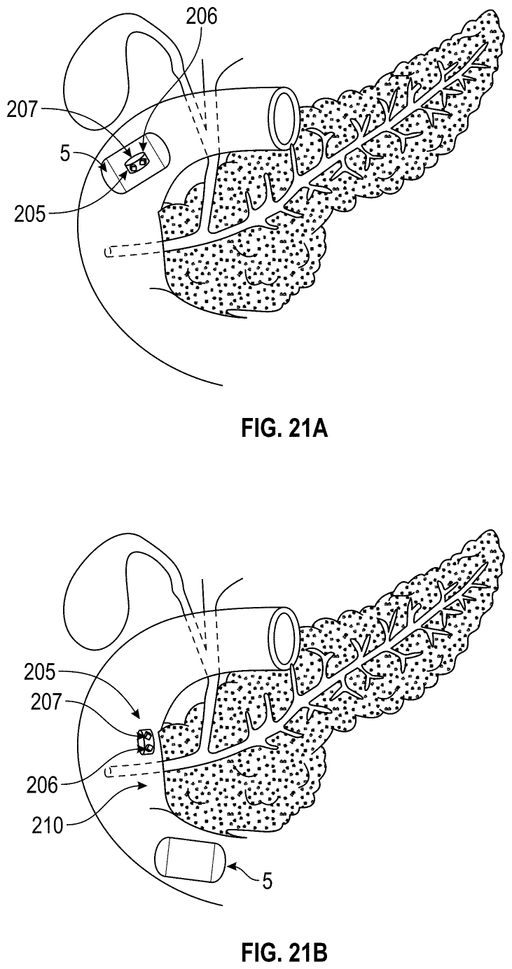

As shown in FIG. 20A, FIG. 20B and FIG. 21 the present disclosure includes an intra-body controllable medical device having one or more imaging systems 205 within (FIG. 20A) or remote (FIG. 20B) to the intra-body controllable medical device. The imaging systems include X-ray radiography, magnetic resonance imaging, medical ultrasonography or ultrasound, confocal microscopy, elastography, optical-coherence tomography, tactile imaging, Raman spectroscopy, thermography and medical digital photography. In some aspects, the imaging systems 205 are configured to travel through the lumen 300 in the intra-body controllable medical device (FIGS. 20A and 20B). In an alternative aspect and referring to FIG. 21A and FIG. 21B, the imaging systems 205 are further configured to be discharged from the intra-body controllable medical device while in the lumen and deposited in a predetermined location in the lumen for ongoing monitoring. As an example, and referring to FIG. 21A, the medical device 5 may be equipped with imaging system 205. The medical device 5 may travel through the small intestine and deposit imaging system 205 adjacent to the Ampulla of Vater 210 (FIG. 21B), as a non-limiting example. The medical device 5 may then continue to travel through the small intestine without the imaging system. The imaging systems 205 may be configured with a storage medium 206 to store images. The imaging systems 205 may further be configured with a transmitting device 207 to transmit real time images to one or more receivers located in other positions in the lumen and those located in other locations and organs in the body (e.g., a human body) and outside of the body.

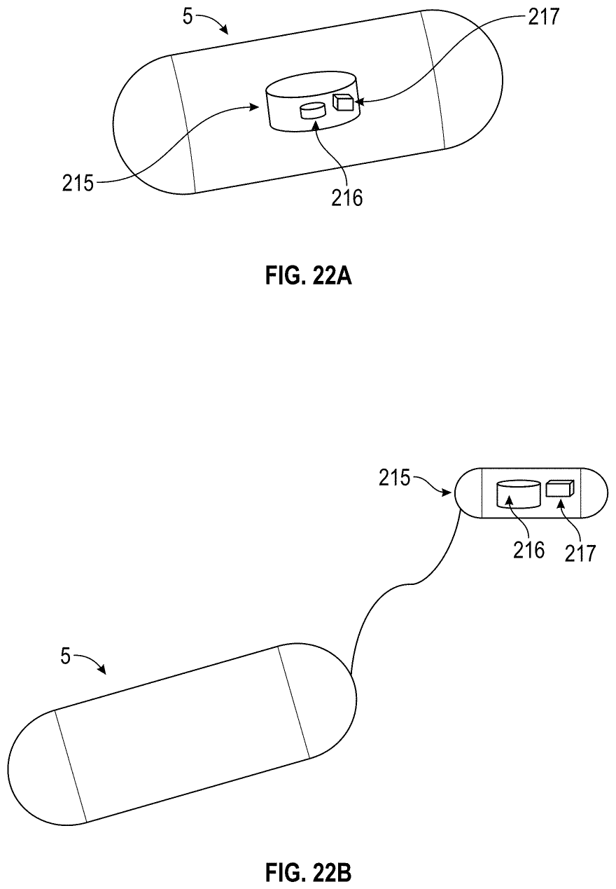

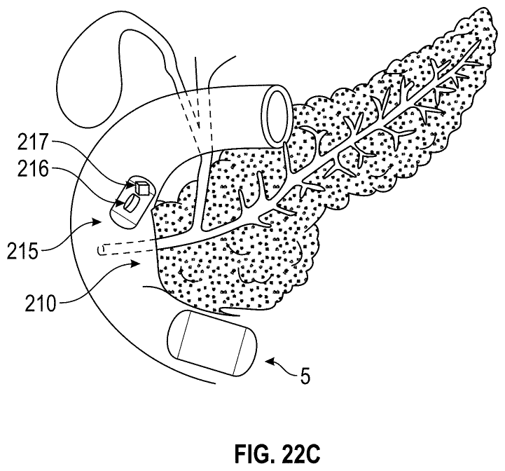

As shown in FIG. 22A, FIG. 22B, and FIG. 22C, the present disclosure includes an intra-body controllable medical device having one or more therapy delivery systems 215 within (FIG. 22A) or remote (FIG. 22B) to the intra-body controllable medical device. The therapy delivery systems 215 include optical-coherence tomography (OCT) guided laser instruments, radiation discharging sources, chemotherapy deploying devices, pharmaceutical and drug deploying devices, ablation devices and photodynamic therapy devices. The therapy delivery systems 215 are configured to travel through the lumen in the intra-body controllable medical device and provide therapy. The therapy delivery systems 215 may further be configured to be discharged from the device while in the lumen 100 and deposited in a predetermined location in the lumen 100 for ongoing therapy delivery (FIG. 22C). The therapy delivery systems 215 may be configured with a storage medium 216 to record time, duration and application location of the therapy. The imaging systems 205 and therapy delivery systems 215 may be further configured with a transmitting device 217 to transmit real time images to one or more receivers located in other positions in the lumen and those located in other locations and organs in the body (e.g., a human body) and outside of the body. The medical device 5 of FIGS. 22A and FIG. 22B may travel through the small intestine and deposit therapy delivery system 215 adjacent to the Ampulla of Vater 210 (FIG. 22C). The medical device 5 may then continue to travel through the small intestine without the imaging system.

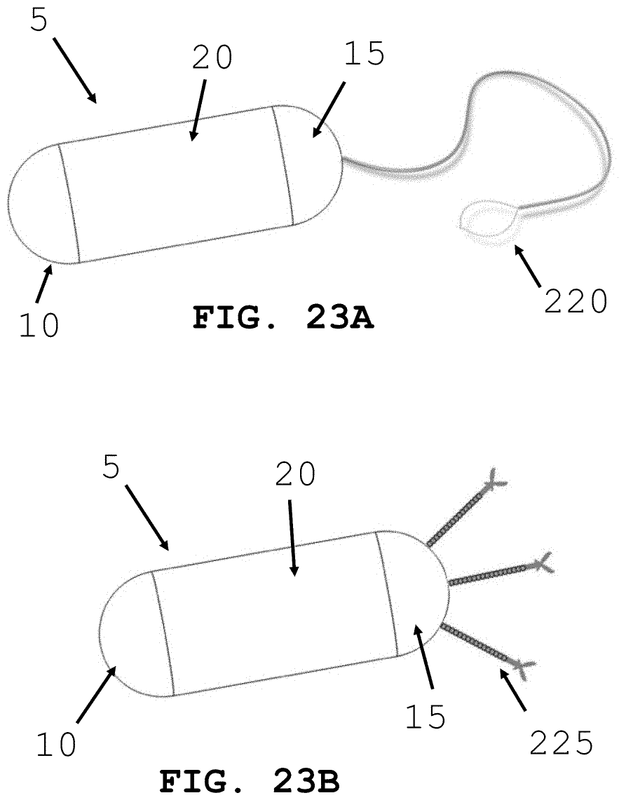

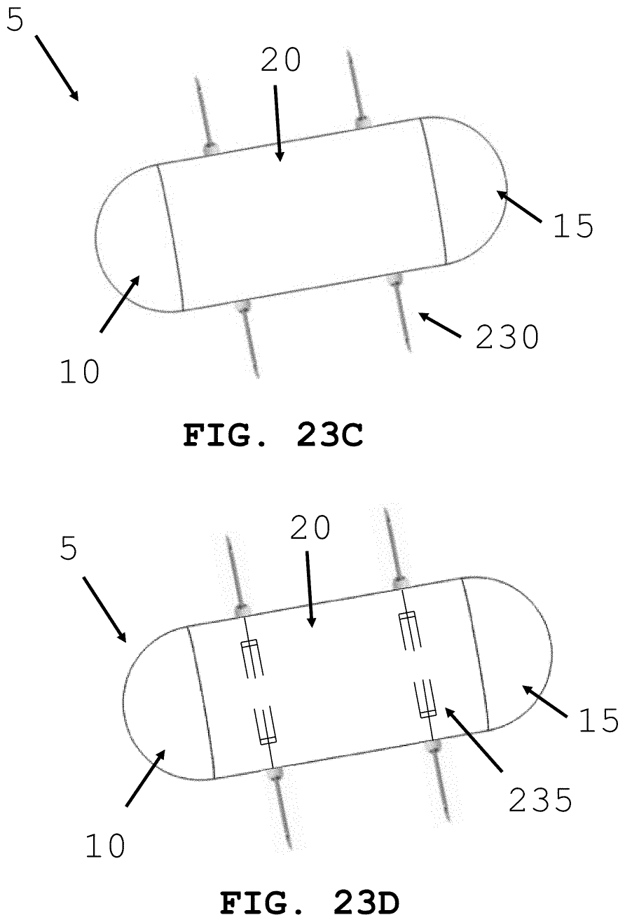

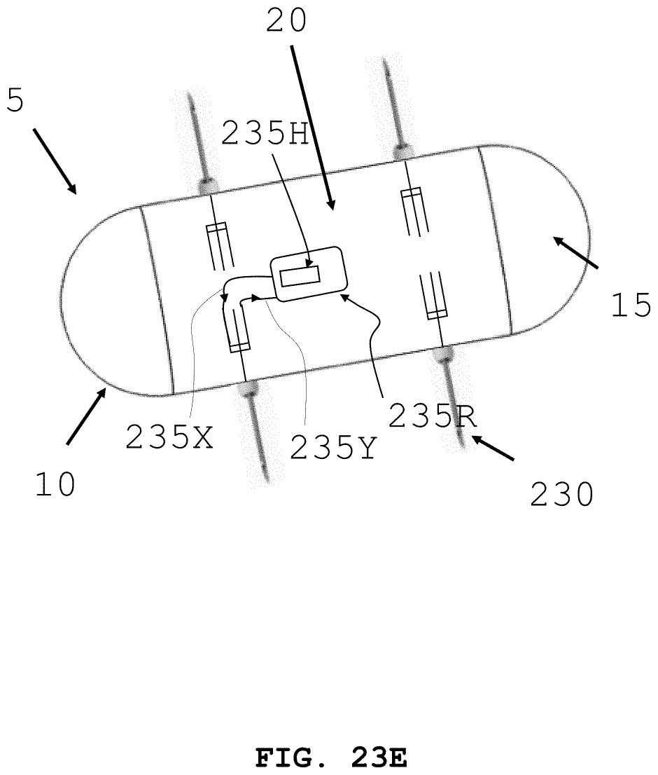

As shown in FIG. 23, the present disclosure includes an intra-body controllable medical device having one or more sample and data gathering systems. The sample gathering systems are configured to obtain tissue biopsies and blood, bone, cells, bone marrow, blood, urine, DNA and fecal samples. The sample gathering devices may include any known in the art including one or more snares 220, forceps 225, needles 230, suction devices 235, and combinations thereof. The data gathering devices may include pH probes, accelerometers, pressure transducers, thermometers, and dimensional measurement systems. The sample and data gathering systems are configured to perform localized testing such as complete blood counts, bone density measurements, acidity testing and turbidity testing. The sample and data gathering systems are configured to take, record and transmit dimensional, angular, velocity and volumetric measurements. The intra-body controllable medical devices contain miniaturized devices for performing the tests and obtaining the data, including miniaturized needle aspiration devices 230, and suction devices 235. The dimensional, angular, velocity and volumetric measurements are acquired by miniaturized devices deployed from the intra-body controllable medical device including ultrasound systems and laser imaging.

As shown in FIG. 23E, intra-body medical device 5 can include a therapy delivery device configured to deliver a therapy, as described in International Patent Application Publication No. WO2020/005816, filed on Jun. 24, 2019, which is incorporated by reference herein in its entirety. As shown, intra-body medical device 5 can include a miniature discharge apparatus such as a needle 230 attached to host structure 20 includes an interior area having a reservoir 235R. The needle 230 may be attached to the reservoir 235R. The reservoir may contain a cold substance such as a liquid, an aqueous solution, a plurality of particulate matter, an isotonic solution, a saline solution, a gel, a slurry (for example, a slurry as described in U.S. Publication No. 20170274011 which is incorporated by reference herein in its entirety), a fat destroying substance and a vasoconstrictor, that can be injected through needle 230 into the body, for example, in the proximity into fat cells (e.g., subcutaneous fat cells and/or visceral fat cells) or other cold therapy receiving region, in order to destroy the fat or otherwise administer the cold therapy. As shown in FIG. 4, the reservoir 235R includes a heat exchanger/heat sink 235H therein. The cold substance is discharged from the heat exchanger/heat sink 235H via a discharge line 235X that is in communication with the discharge device (e.g., needle) 230 for circulating or discharging the cold substance therefrom. Alternatively, the needle may be closed and instead of injecting the cold substance (e.g., cold fluid or slurry) into the fat or other cold therapy receiving region, the cold slurry or fluid may be circulated, in a closed loop manner, through the needle to generate localized cooling in the cold therapy receiving region. For example, as shown in FIG. 4, the cold substance is returned to the heat exchanger/heat sink 235H in the reservoir 235R disposed in the host structure 20, via a return line 235Y, for further cooling in the heat exchanger/heat sink 235H.

The needle 230 may be sized so as not to create scars. In some aspects, the needle is smaller than a 16-gauge needle. In some aspects, the needle may be a “micro” needle—with a diameter less than 1.0 mm. In addition to being able to provide cooling through the needle, micro needle may create small holes known as micro-conduits that generate minimal damage to tissue such as the epidermis. This process can lead to the generation of growth factors which stimulate the production of collagen and elastin in the papillary layer of the dermis. These micro-conduits may be used to treat scarring and wrinkles, enable skin rejuvenation and brightening, improve the appearance of skin (anti-ageing), treat disorders of pigmentation, hyperhidrosis, striae, induce collagen synthesis under the epidermis, treat hair pathology as it may stimulate stem cells in the dermal papilla, increase blood flow to hair follicles, and recruit growth factors and signaling pathways which induce hair restoration, and fill in fine lines and plump the skin. Alternatively, the heat exchanger/heat sink 235H may be used to cool the surface of intra-body controllable medical device 5.

Accordingly, in aspects of the present disclosure, a medical device for intra-body conveyance is directed to administering cold therapy within a body (e.g., a human body). The medical device includes a host structure that has a reservoir for containing a cold substance (e.g., a liquid, an aqueous solution, a plurality of particulate matter, an isotonic solution, a saline solution, a gel, a slurry, a fat destroying substance and a vasoconstrictor). The medical device includes one or more delivery apparatuses (e.g., a needle) in communication with the host structure, for delivery the cold substance for administering cold therapy within a body.

In some aspects, the delivery apparatus or needle is configured to inject the cold fluid inside the body. In other aspects, the delivery apparatus or needle is configured to circulate the cold substance to a predetermined cold therapy receiving region and returns the cold substance to the host structure for further cooling. In certain aspects, the delivery apparatus or needle is a microneedle having a diameter of less than about 1.0 mm.

In some aspects, the medical device is in communication with at least one repository. The at least one repository includes at least one of a heat sink, a heat exchanger, a chemical reactor and a storage vessel.

In some aspects, the host structure includes at least one of a clinically inert material, a sterilizable material, an elastomeric material, a chemically reactive material, a chemically inert material, a disintegrable material, a dissolvable material, a collapsible material and a material having physical and chemical properties to withstand exposure to bodily fluids for a predetermined period of time.

In another aspect of the present invention, a plurality of medical devices is in communication with at least one repository. The at least one repository includes at least one of a heat sink, a heat exchanger, a chemical reactor and a storage vessel. At least one the plurality of medical devices includes a host structure having a reservoir for containing a cold fluid and at least one needle for delivery of cold therapy within a body. The at least one repository can be positioned in at least one of inside the body or outside the body. In some aspects, the at least one repository is connected to at least one of the plurality of medical devices by at least one of a network of conduits, tubes, cannulas, capillaries, heat conducting materials and ducts.

In another aspect, a method for using the medical device is directed to using a cold fluid to effect destruction of fat cells with a body.

The present disclosure also includes a method for using a medical devices for administering cold therapy in a body. The method includes disposing the medical device inside the body, proximate a cold therapy receiving site. A cold substance is discharged or circulated into the receiving site for a predetermined time, thereby administering the cold therapy within the body. In certain aspects, the method of using the medical devices is directed to use for treatment of scars, wrinkles, disorders of pigmentation, hyperhidrosis, destroying subcutaneous fat cells, destroying visceral fat cells, inducing collagen synthesis and/or inducing hair restoration.

In certain aspects, a method of providing therapeutic treatment to a patient includes inserting a medical device into a patient's body lumen; navigating the medical device to a specific site in need of a site-specific cold therapy; and delivering the site-specific cold therapy in proximity to the site in need of the therapy. The aspects described in connection with FIGS. 23A-23E are exemplary and do not limit the scope of the present disclosure.

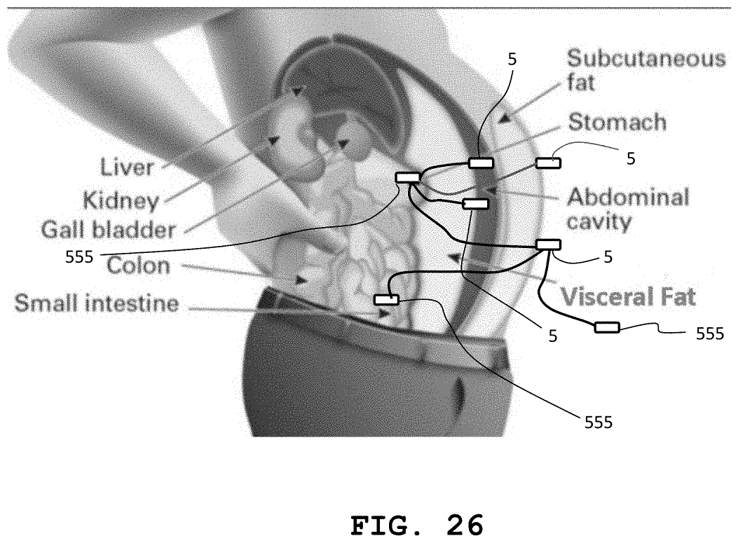

As shown in FIG. 26, intra-body medical device 5 or an interactive group thereof may be used to deliver cold therapy within the patient. A plurality of medical devices (5) may be in communication with one or more repositories (555). The repositories 555 include heat sinks, heat exchangers, chemical reactors and/or storage vessels. The plurality of medical devices has a cooling system and/or a material discharge system disposed therein or thereon. The repositories 555 are positioned intra body (i.e., inside the human body) and/or outside the human body. The intra-body medical devices 5 are shown connected to each other and the repositories 555 by a network of conduits (e.g., tubes, cannulas, capillaries, heat conducting materials and ducts).

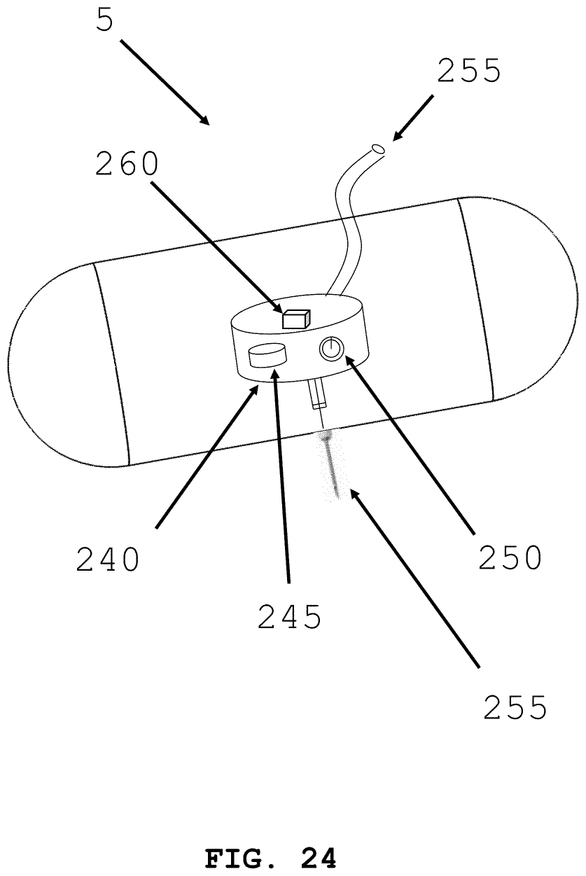

As shown in FIG. 24, the present disclosure includes an intra-body controllable medical device having one or more material dispensing systems. The material dispensing systems 240 are equipped with storage compartments 245 for storing and dispensing payloads including medication, liquids, powders, chemically reactive agents and radiation emitting sources and recording and tracking the location of the payloads before and after the dispensing operation. The material dispensing systems include actuators, pumps, compressors, nozzles, flow control devices 250 including valves and orifices, injection and piercing devices 255 and dose measuring and recording devices 260.

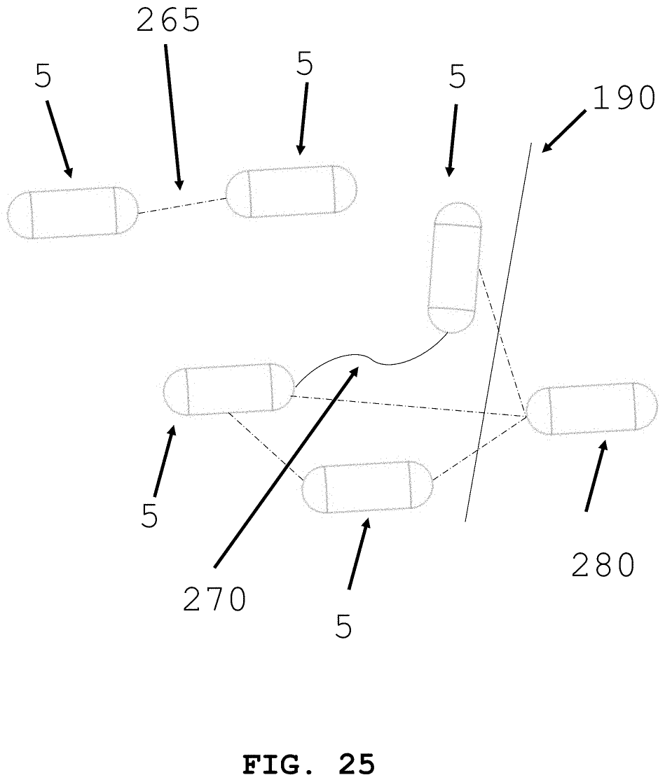

As shown in FIG. 25, the present disclosure includes an interactive group of intra-body controllable medical devices. The interactive group of devices includes two or more devices 5 that are in communication with one another and/or an external computer-based control system. The two or more intra-body controllable medical devices are configured to cooperate with one another to distribute components such as power supplies, medical devices, storage compartments and auxiliary devices among the intra-body controllable medical devices so that the intra-body controllable medical devices operate together as a group to accomplish the intended functional operations. The interactive group of intra-body controllable medical devices is configured to operate collectively as a swarm of a plurality of intra-body controllable medical devices that if deployed individually would not be as effective in undertaking the intended medical procedure or other functional operation. The interactive group of intra-body controllable medical devices includes tethering 270 or towing devices (e.g., winches) between intra-body controllable medical devices to assist in propulsion of the intra-body controllable medical devices through the lumens. Additionally, the intra-body medical devices may communicate wirelessly 265 between devices. Intra-body medical devices may communicate with a receiver or controller 280 located outside the body 190. Intra-body medical device 5 may operate like a drone, communicating and being controlled by an operator in the same room or in a different location from the patient. Furthermore, when contemplating a swarm of devices, two or more intra-body controllable medical devices 5 may be deployed. A first intra-body medical device 5 may leave the swarm group and navigate to a region of interest. This device may perform a first task and communicate back to the other devices in the swarm and direct a second device 5 to navigate to the first device 5. Second device 5 may be selected from a number of devices in the swarm because of its particular capabilities (e.g., second device 5 may have an additional battery, an imaging system, a therapy system, a sample and data gathering system, and/or a material dispensing system). Second device 5 may transfer capabilities to first device 5 or second device 5 may perform a task related to its specific capabilities. This serial communication and deployment of devices from the swarm may continue until the desired procedure is completed.

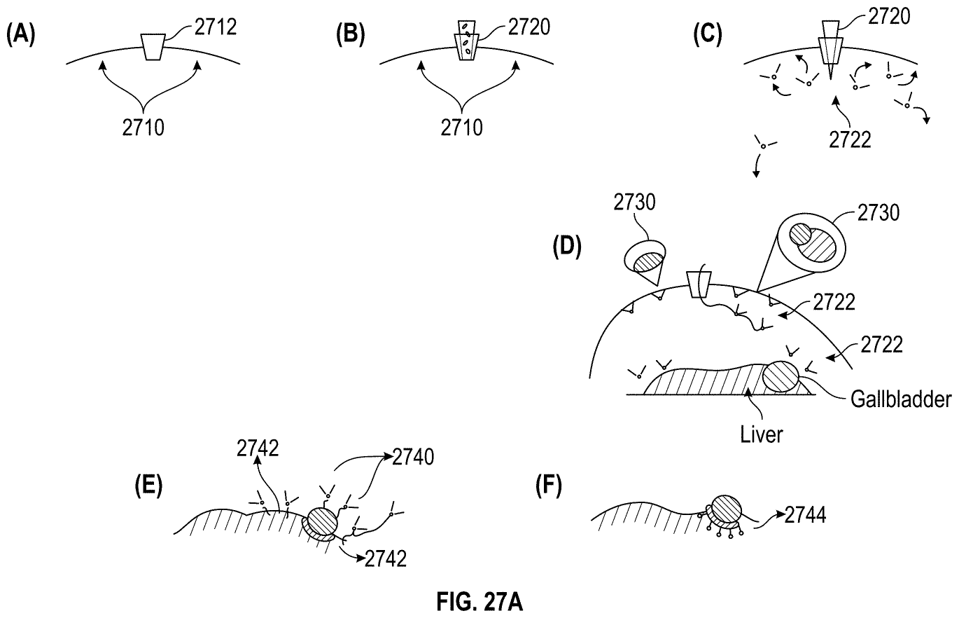

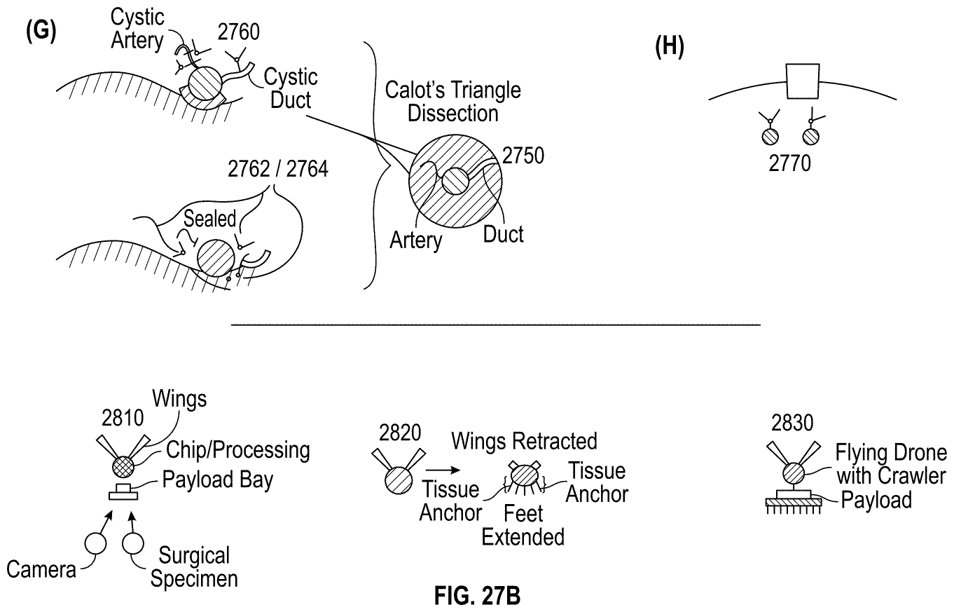

As shown in FIGS. 27A and 27B, an interactive group of intra-body medical devices may be used in a laparoscopic cholecystectomy procedure within a patient. The group of intra-body medical devices may be referred to as a swarm of devices/drones. In accordance with aspects of the present disclosure, a swarm can provide the capability of performing certain operations in a parallel manner.

The operating environment for the procedure may provide visualization technology, such as omni vision, real-time three-dimensional visualization, and/or overlay of pre-operative images and planning information. In various aspects, the group of intra-body medical devices may provide SLAM-based vision stitching to visualize a compound image.

With continuing reference to FIGS. 27A and 27B, the operation can begin with positioning the patient for gaining access to the surgical site, which may include positioning a patient in a head-up or a left-side down position, and insufflating the abdomen 2710. When a patient is in the appropriate position, in step (A), laparoscopic ports and/or trocars 2712 are inserted into the patient. In various aspects, the ports 2712 can include one or more of 10 mm umbilical port, 10 mm epigastric port, 5 mm port directly over the gallbladder, and/or 5 mm port over the mid-clavicular line. A surgeon typically can obtain a first view once a camera is inserted proximate the liver and gallbladder. However, such a view is typically not ideal, as there is usually some fat connected to the gallbladder bed and the liver behind. Rather, in accordance with aspects of the present disclosure, in steps (B)-(D), a swarm of devices or drones 2722 from a cartridge 2720 can be deployed through a port or trocar 2712 and into the insufflated abdomen 2710, to capture images 2730 to provide a 360-degree view to orientate the surgeons from the first moments of visualization.

Once a surgeon has visualization, the procedure involves retracting the gallbladder. The surgeon can grasp the gallbladder by using a “gallbladder grasper” and then retracting the gallbladder. Then, the liver is retracted, and fatty adhesions are dissected. In various aspects, a swarm of devices/drones acting concurrently could perform these procedures in parallel, as opposed to one at a time, by retracting the gallbladder 2740 while retracting the liver 2742 at the same time, in step (E), to give the surgeon or other drones an access to dissect the fat 2744, in step (F). In various aspects, rather than having a surgeon use a “gallbladder grasper” and securing it in place using a towel outside of patient or using an assistant, various drones can maintain position of the gallbladder in-situ, which can mitigate safety and/or logistic concerns associated with this procedure.

Next, the procedure involves dissecting Calot's triangle. The gallbladder is retracted anteriorly and superiorly. There is then blunt dissection around the cystic duct and artery where the peritoneum is overlying. Diathermy is not conducted here due to important structures such as the bile duct, which is susceptible to thermal injury. During these procedures, multiple windows are created to provide further access. Rather than using irrigation/suction device for creating these windows, a swarm of drones could make create such access windows concurrently. Should irrigation/suction be needed, various drones can perform such irrigation and/or suction. During these procedures, the patient anatomy may be difficult to distinguish, as ligation of cystic artery and ducts is required, but these two are situated in extremely close proximity to other important structures such as the bile duct and duodenum. In various aspects, a swarm of imaging drones 2750 with AI enabled software can assist a surgeon with visualizing the gallbladder, cystic artery, and ducts. The windows are created behind the cystic duct.

Next, the procedure involves identifying and ligating cystic duct and artery. The cystic artery is clipped using three clips, and various drones can be deployed for clipping purposes 2760, decreasing risk of incorrect clip placement. A cut is made distally to the second clip, and the stump of artery left behind has two clips. The third clip is in close proximity to the gallbladder where it is subsequently cut using laparoscopic scissors. In various aspects, drones can be used for cutting 2762 as soon as the artery is clipped at both ends, to save time. The cystic duct is clipped, similar to artery.

Next, the procedure involves resecting and removing the gallbladder, which is now possible as the cystic duct and cystic artery have been ligated and transected. Hook diathermy is used and the appearance of the loose areola tissue is used as the anatomical plane for dissection. Hook diathermy is used moving away from important structures. During this procedure, there is a lot of bleeding in the gallbladder bed, as it is very close to the liver parenchyma. The gallbladder is dissected laterally from the heel of the gallbladder bed off the liver. A bird bag is used to place the dissected gallbladder where it rests while the area is inspected for bleeding. Suction/irrigation is used to double check bleeding from the liver. In various aspects, a swarm of drones can check for bleeding 2764 concurrently with the gallbladder dissection 2762, rather than check for bleeding after the dissection, which prevents the need of leaving the gallbladder in-situ while suction and irrigation are done to check for bleeding. A swarm of imaging drones 2770 can check the entire landscape to ensure no complications have arisen.

Next, the procedure involves closing the surgical site, including closing the 10 mm port sites and 5 mm port sites. The 10 mm port sites may involve deep and/or superficial closure, and the 5 mm port sites may involve subcuticular sutures or glue.

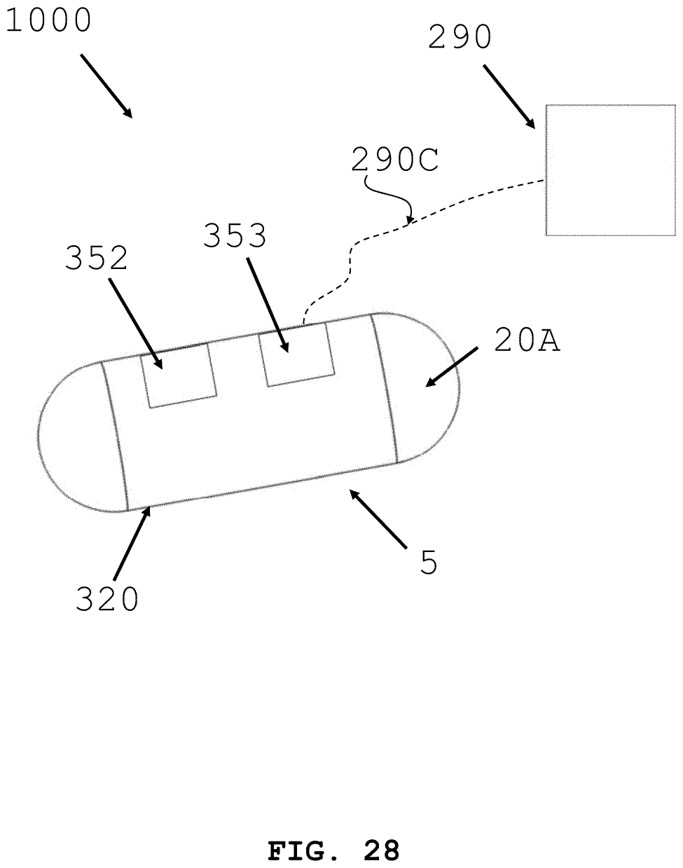

In accordance with aspects of the present disclosure, various complications may arise during or after the procedure of FIGS. 27A and 28B, including bleeding from cystic artery stump or liver parenchyma or liver bed, infection, bile leak from the cystic duct stump, bile duct damage that may require extensive bile duct reconstruction, damage to other structures, venous thromboembolism, and/or anesthetic complications. In various aspects, some or all of these complications may be monitored in parallel using a swarm of drones 2770.

The drones illustrated in FIG. 27B may include drones designed for flying, swimming, and/or crawling on surfaces inside a body. In an exemplary configuration 2810 for flying and/or swimming, a drone can include wings, chip/processing, and a payload bay, which may contain a camera and/or a surgical specimen. In an exemplary configuration 2820 for flying and/or crawling, a convertible drone can convert from a flying drone to a crawling drone by retracting wings and extending feet into a tissue surface to anchor the drone to issue for crawling. In an exemplary configuration 2830, the drone may be a flying drone that carries a crawler as a payload. In the various configurations, biocompatible materials are used. The illustrated and disclosed configurations are exemplary, and variations are contemplated to be within the scope of the present disclosure.

The illustrated procedure of FIGS. 27A and 27B is exemplary, and the parallelization of operations using a swarm of intra-body devices can be applied to other procedures as well. As an example, a swarm of devices operating in parallel can be applied to surgical site preparation and/or tissue staging operations, including tissue hold, stretch, and/or motion compensation. In another example, a swarm of devices operating in parallel can be applied to surgical procedures such as suction, cutting, dissection, blood loss management, delivery of a therapy and/or tissue sealing. As another example, a swarm of devices operating in parallel can be applied to tissue sampling procedures, such as biopsy, tissue characterization, perfusion, and/or vascularity. In another example, a swarm of devices operating in parallel can be applied to drug delivery procedures, such as localized drug delivery for therapy and/or dye replacement. As another example, a swarm of devices operating in parallel can be applied to post-operative surveillance procedures, such as anchoring near a surgical site to aid in monitoring pathology and healing. In another example, a swarm of devices operating in parallel can be applied to implant delivery procedures, such as smart implant construction. Other applications are contemplated to be usable with a swarm of intra-body devices operating in parallel.

In aspects of the present disclosure, artificial intelligence and machine learning (hereinafter collectively referred to as “AI”) can be employed in guiding and informing the actions of the intra-body controllable medical device 5, as described in International Application Publication No. WO2020/005815 filed on Jun. 24, 2019, which is incorporated by reference herein in its entirety. AI is employed to enable the intra-body controllable medical device 5 to make diagnostic decisions, provide therapy, and alert the physician of a pathology.

AI is employed for reviewing the large volumes of data that the intra-body controllable medical device 5 generates. Depending on its configuration, and as described in previous figures, the device 5 may include different imaging, sensor, probe, and sample technologies. Imaging at the cellular level generates significant amounts of data, which is far too much for a physician to analyze in real-time. AI allows for the analysis of data transmitted from the device 5 and facilitates clinically relevant decisions.

As shown in FIG. 28, the medical device 5 utilized with AI is encompassed in a medical system 1000. The medical system 1000 includes the medical device 5 for intra-body conveyance, and thus, the medical device 5 of the medical system 1000 may be deployed in a body of a patient. It is contemplated that the medical device 5 used in the medical system 1000 is of any configuration according to the aspects described and illustrated herein, however, the medical device has a host structure 320 defining an interior area 20A. The medical device 5 of the medical system 1000 includes at least one data gathering system, such as cameras, pH probes, accelerometers, pressure transducers, thermometers, and dimensional measurement systems, and combinations of any of the foregoing. In some aspects, medical device 5 can also include a sample gathering system as described herein.

The medical device 5 of the medical system 1000 includes at least one means for communication, such as, for example, communication devices (e.g., transmitters 352 and receivers 353) and a processing device 290, for example a processing device that is external to the body or a processing device that is internal to the body 190 or medical device 5. The means for communication transmit data from the data gathering system to the external processing device 290 (e.g., via a communication path 290C such as a wireless communication link), which is configured to receive and analyze the transmitted data from the medical device 5. The processing device 290 implements or applies a set of instructions (also referred to as “AI instructions”, an “AI algorithm” and/or “AI technology”) that analyzes the data received from the medical device 5 and provides a user with a diagnosis and/or a recommendation for treatment.

The medical system 1000 is configured to store information and point physicians to the location of key relevant findings in a patient's body in a concise, easily discernable, and actionable format, including the use of machine learned and AI algorithms programed as software code on a computer processor located within or remote to the medical device 5. In addition, the recommendations provided by the AI instructions employed in the medical system 1000 can overlay a patient's own previous medical record to provide a more personalized treatment plan. AI technology employed in the medical system 1000 can help reduce the time between scanning, diagnosing and treating a patient. AI technology employed in the medical system 1000 can play a key role in offering faster treatment options, reducing the number of procedures a patient may undergo, and decreasing the overall financial burden on the healthcare system. AI technology employed in the medical system 1000 can reduce diagnostic and therapeutic errors that are inevitable in human clinical practice. AI technology employed in the medical system 1000 extracts useful information from larger patient population and can assist physicians and other healthcare practitioners in making real-time inferences for health risk alerts and health outcome prediction. AI technology employed in the medical system 1000 can also assist physicians by providing up-to-date medical information and best clinical practices to better inform proper patient care.

The medical system 1000 and the medical device 5 is used in a method of diagnosing or treating at least one anomaly in a patient. The method includes placing at least one medical device 5 according to any aspect described herein into a lumen or an orifice of a patient. The medical device 5 is placed into a lumen or an orifice of a patient as described herein and as is known in the medical art. Once the medical device 5 is placed in a patient, data about the patient is collected with the medical device. The data collected by the medical device can include the data described above, which includes, but is not limited to, images, pH, size, etc. The data is collected by the sensors and data/sample gathering systems described herein. That collected data is then transmitted from the medical device 5 to a processing device 290 such as a processing device that is external to the body 190 or a processing device that is internal to the body or the medical device 5. As discussed above, the data is transmitted through devices in the medical device 5 and the processing device 290 either wirelessly or through hard wired connections. One received by the processing device 290 instructions provided to or stored on the external processing device are applied to the data received from the medical device 5. Application of the instructions analyzes the data and the analyzed data is used to diagnosis or treat the patient.

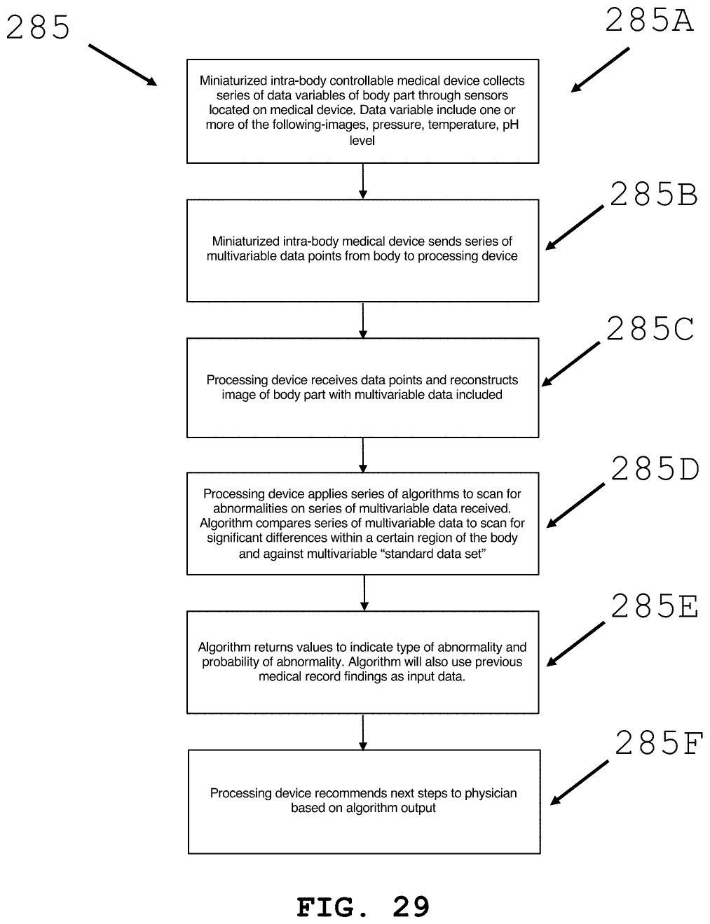

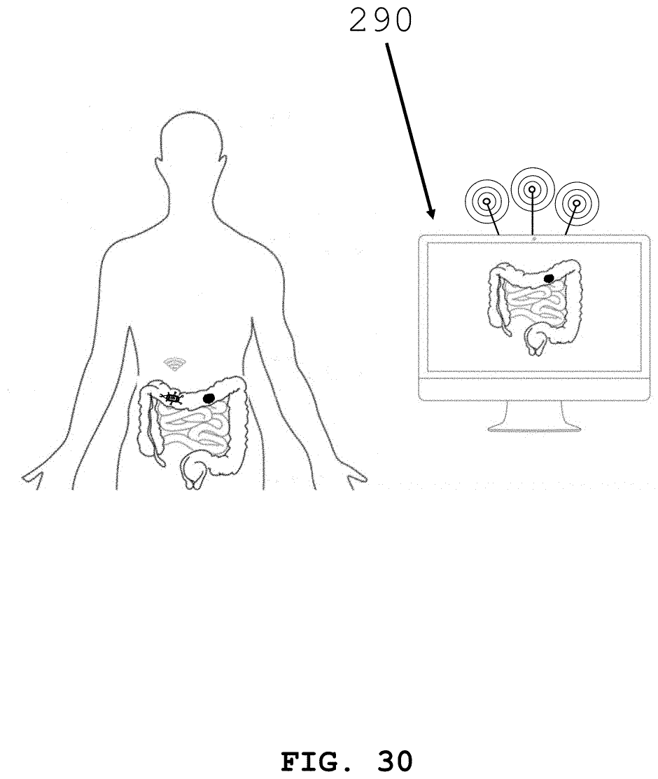

FIG. 29 shows a flow chart 285 of steps that implemented the AI algorithm for analyzing and making diagnostic and therapeutic decisions by utilizing data collected by the intra-body controllable medical device 5. As shown in FIG. 29, in step 285A, the miniaturized intra-body controllable medical device 5 collects a series of data using the data gathering system(s), e.g., sensors and imaging components. The data may include images, temperature, pH, or pressure. In real time, in step 285B, the intra-body controllable medical device transmits the data to a processing device 290 (FIGS. 28 and 30). As shown in FIGS. 28 and 30, the processing device 290 is external to the body of the patient (i.e., an external processing device). The external processing device 290 is any type of processing device configured to receive data from the medical device 5. In some aspects, as shown by dashed lines in FIGS. 28 and 30, the external processing device 290 is in wireless communication with the medical device 5. In another aspect, the external processing device 290 is connected to the medical device with one or more wires. In some aspects, the external processing device 290 is a cloud computer, a local computer terminal, or a device carried by the patient. In some aspects, the external processing device 290 is connected to one or more graphical user interfaces for display of information to a user (e.g., physician, nurse, patient). In some aspects, the external processing device 290 is connected to at least one of the internet, a server, and an external database. It is contemplated that more than one medical device 5 can communicate with a single external processing device 290 and that more than one external processing device 290 can communicate a single medical device 5.

Referring still to FIG. 29, in step 285C, the processing device 290 receives the data from the medical device 5, e.g., by transmission from at least one means for communication 353 and the AI algorithm stored in a computer processor disposed in the medical device, received for sources external to the medical device 5 (e.g., another medical device, the internet, a computer located outside of the body, and in step 285D, the processing device 290 compares the data received from the medical device 5 to a master data set stored on the processing device 290. The master data set contains information on both normal and pathologic samples. In steps 285E and 285F, when the data received from the medical device 5 lies outside the normal samples in the master data set, the instructions according to the AI algorithm marks the data as abnormal and compares the data to other pathologic samples in order to make a diagnosis and recommends next steps of treatment based on the application of the AI algorithm to the data received from the medical device 5 and compared to the master data set.

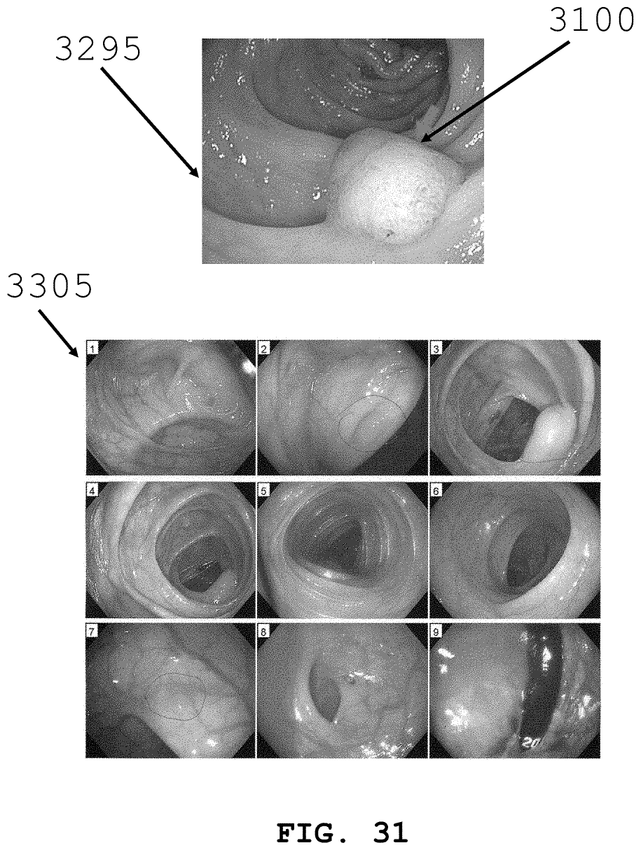

As seen in FIG. 31, and as an example using images, intra-body controllable medical device 5 takes an image 3295 with a camera installed in or on the medical device 5. Image 3295 is transmitted via means for communication 353 to the processing device 290. At processing device 290, the AI algorithm processes the image in accordance with the flow chart 285 shown in FIG. 29. Image 3295 is compared to a database of other images 3305. A region of interest 3100 in the image 3295 is identified by the AI algorithm and flagged as abnormal by the AI algorithm. The region of interest 3100 is identified by the AI algorithm stored on the processing device 290 as a colonic polyp.

Depending on the diagnosis, the processing device 290 sends a signal back to the intra-body controllable medical device 5 to perform an action (i.e., a biopsy, additional imaging, etc.). Alternatively, the processing device 290 may request another intra-body controllable medical device 5 be deployed or summoned to the site of the abnormality.

The present disclosure is directed to configurations, materials and structures for intra-body controllable medical devices. In some aspects, the medical devices can be disposable, disintegrable and selectively collapsible intra-body controllable medical devices. The intra-body controllable medical devices can be manufactured of a material such as an elastomer (e.g., nitrile) that can expand and contract, for example, by inflating and deflating them. The intra-body controllable medical devices can be manufactured from a biodegradable, disintegrable or dissolvable material, including paper, starches, biodegradable material such as gelatin or collagen and/or synthetic natural polymers. The collapsible intra-body controllable medical devices can be configured to be flattened, extruded, stretched or disassembled in the lumen. Thus, the intra-body controllable medical devices can be disposed of in the lumen or via discharge therefrom without the need to recover the intra-body controllable medical devices for analysis, inspection or future use.

Materials for manufacture of an intra-body controllable medical device can be clinically inert, sterilizable, elastomeric (e.g., contractible and expandable), chemically reactive, chemically inert, dissolvable, collapsible and have physical and chemical properties to withstand exposure to bodily fluids for precise predetermined periods of time. Such materials include but are not limited to polymers, metallic alloys, shape memory polymers, shape memory metal alloys, shape memory ceramics, composites, silicones, thermoplastic polyurethane-based materials, excipients, zeolite adsorbents and styrene-butadiene rubbers (SBR). Materials may further include biodegradable materials such as paper, starches, biodegradable material such as gelatin or collagen.

The present disclosure is directed to methods for using intra-body controllable medical devices in the medical field and in particular for use in administering medications and therapy, deploying medical devices, imaging and surgery. The methods for using intra-body controllable medical devices includes applications in the gastro/intestinal tract (e.g. colonoscopy), urology applications, in the lungs, bladder, abdomen, pelvic cavity, dorsal cavity, nasal and reproductive systems, in performing Transurethral Resection of Bladder Tumors (TURBT), Transurethral Resection of the Prostate (TURP) and transrectal prostate ultrasound, biopsy, and radiation treatment. The methods for using intra-body controllable medical devices include use in procedural environments, operatory/surgical procedures, ambulatory/out-patient procedures and unobtrusive normal routine living.

Although the present technology has been disclosed and described with reference to certain aspects thereof, it should be noted that other variations and modifications may be made, and it is intended that the following claims cover the variations and modifications within the true scope of the disclosure.

What is claimed is:

1. A system for assisting with a surgical procedure, comprising:
a swarm of medical devices sized to be wholly deployed within a surgical site of a patient, the swarm of medical devices configured to operate concurrently within the patient to assist a surgeon perform a surgical procedure in the patient,
the swarm of medical devices including:
a first medical device sized to be wholly deployed within the surgical site and including an imaging system configured to capture a view of at least a portion of a surgical site and to communicate the captured view, and
a second medical device sized to be wholly deployed within the surgical site and including at least one of: a retracting device, an irrigation device, a suction device, a clipping device, a therapy delivery device, or a cutting device.

2. The system of claim 1, wherein the swarm of medical devices includes a plurality of medical devices having imaging systems, the plurality of medical devices including the first medical device,
wherein the plurality of medical devices is configured to cooperate to capture a 360-degree view of the surgical site.

3. The system of claim 1, wherein the second medical device includes the retracting device,
the swarm of medical devices including a third medical device including a retracting device,
wherein the second medical device and the third medical device operate to assist the surgeon perform the surgical procedure by concurrently retracting different portions of the surgical site using their respective retracting devices.

4. The system of claim 3, wherein the second medical device is configured to retract a gallbladder and the third medical device is configured to concurrently retract a liver.

5. The system of claim 1, wherein the second medical device includes at least one of a suction device or an irrigation device,
wherein the second medical device is configured to assist the surgeon perform a dissection of Calot's triangle by creating an access window using at least one of: the suction device or the irrigation device.

6. The system of claim 5, wherein the second medical device is configured to create the access window behind cystic ducts.

7. The system of claim 1, wherein the first medical device is configured to assist the surgeon perform the surgical procedure by capturing a view of at least one of: a gallbladder, cystic artery, or cystic ducts.

8. The system of claim 1, wherein the second medical device includes the clipping device and is configured to assist the surgeon perform the surgical procedure by clipping a cystic artery.

9. The system of claim 8, wherein the swarm of medical devices includes a plurality of medical devices having clipping devices, the plurality of medical devices including the second medical device,
wherein the plurality of medical devices is configured to cooperate to apply multiple clips to at least one of: the cystic artery or a cystic duct.

10. The system of claim 9, further comprising a third medical device including a cutting device, the third medical device configured to cut at least one of the cystic artery or the cystic duct between two of the multiple clips.

11. The system of claim 1, wherein the swarm of medical devices includes a plurality of medical devices configured to cooperate to identify surgical complications in the surgical site.

12. The system of claim 11, wherein the plurality of medical devices is configured to cooperate to identify at least one of: bleeding or infection.

13. The system of claim 11, wherein the plurality of medical devices is configured to cooperate to identify at least one of: bile leak or bile duct damage that may require extensive bile duct reconstruction.

14. The system of claim 1, wherein at least one of the first medical device or the second medical device includes a pull device which includes a retractable anchor and a tether, wherein propulsion is generated by retracting the tether, thereby pulling at least one of the first medical device to the second medical device to a predetermined position.

15. The system of claim 1, wherein at least one of the first medical device or the second medical device includes a push device which includes a push rod adjacent to a fixed structure, wherein propulsion is generated by advancing the push rod, thereby pushing at least one of the first medical device or the second medical device to a predetermined position.

16. The system of claim 1, wherein at least one of the first medical device or the second medical device includes at least one of a pull device having magnets or a push device having magnets.

17. The system of claim 1, wherein at least one of the first medical device or the second medical device comprises:
an arrangement of inflating and deflating balloons; and
a controller for controlling flow of a fluid to and from the balloons causing the balloons to expand and deflate, thereby creating propulsion of at least one of the first medical device or the second medical device.

18. The system of claim 1, wherein the first medical device includes a first power supply and the second medical device includes a second power supply,
the system further comprising a tether configured to connect the first power supply with the second power supply and configured to allow transfer of power between the first power supply and the second power supply through the tether.

19. A method for assisting with a surgical procedure, comprising:
deploying a swarm of medical devices sized to be wholly deployed within a surgical site of a patient, the swarm of medical devices including:
a first medical device sized to be wholly deployed within the surgical site and including an imaging system configured to capture a view of at least a portion of a surgical site and to communicate the captured view, and
a second medical device sized to be wholly deployed within the surgical site and including at least one of: a retracting device, an irrigation device, a suction device, a clipping device, a therapy delivery device, or a cutting device; and
concurrently operating the swarm of medical devices within the patient to assist a surgeon perform a surgical procedure in the patient.

20. The method of claim 19, wherein the swarm of medical devices includes a plurality of medical devices having imaging systems, the plurality of medical devices including the first medical device,
the method further comprising operating the plurality of medical devices cooperatively to capture a 360-degree view of the surgical site.

21. The method of claim 19, wherein the second medical device includes the retracting device,
the swarm of medical devices including a third medical device including a retracting device,
the method further comprising operating the second medical device and the third medical device to assist the surgeon perform the surgical procedure by concurrently retracting different portions of the surgical site using their respective retracting devices.

22. The method of claim 19, wherein the second medical device includes at least one of a suction device or an irrigation device,
the method further comprising operating the second medical device to assist the surgeon perform a dissection of Calot's triangle by creating an access window using at least one of: the suction device or the irrigation device.

23. The method of claim 19, wherein the swarm of medical devices includes a plurality of medical devices having clipping devices,
the method further comprising cooperatively operating the plurality of medical devices to apply multiple clips to at least one of: the cystic artery or a cystic duct.

24. The method of claim 23, wherein the swarm of medical devices includes a third medical device having a cutting device,
the method further comprising operating the third medical device to cut at least one of the cystic artery or the cystic duct between two of the multiple clips.

25. The method of claim 19, further comprising cooperatively operating the swarm of medical devices cooperate to identify surgical complications in the surgical site.

26. The method of claim 19, wherein at least one of the first medical device or the second medical device includes a pull device which includes a retractable anchor and a tether,
the method further comprising pulling at least one of the first medical device to the second medical device to a predetermined position by retracting the tether.

27. The method of claim 19, wherein at least one of the first medical device or the second medical device includes a push device which includes a push rod adjacent to a fixed structure,
the method further comprising pushing at least one of the first medical device or the second medical device to a predetermined position by advancing the push rod.

28. The method of claim 19, wherein at least one of the first medical device or the second medical device includes at least one of a pull device having magnets or a push device having magnets.

29. The method of claim 19, wherein at least one of the first medical device or the second medical device comprises:
an arrangement of inflating and deflating balloons; and
a controller for controlling flow of a fluid to and from the balloons causing the balloons to expand and deflate, thereby creating propulsion of at least one of the first medical device or the second medical device.

30. The method of claim 19, wherein the first medical device includes a first power supply and the second medical device includes a second power supply, wherein a tether connects the first power supply with the second power supply,
the method further comprising allowing transfer of power between the first power supply and the second power supply through the tether.
